(19) United States
(12) Patent Application Publication
(10) Pub. No.: US 2018/0303346 A2
(43) Pub. Date: Oct. 25, 2018

(54) SYSTEM AND METHOD FOR STORAGE OF CONTAINERS

(71) Applicant: Israel Aerospace Industries Ltd., LOD (IL)

(72) Inventors: Hanan Lepek, Jerusalem (IL); Boris Mogilnsky, Rehovot (IL)

(21) Appl. No.: 15/112,265

(22) PCT Filed: Jan. 18, 2015
(86) PCT No.: PCT/IL2015/050057

§ 371 Date: Jul. 18, 2016

(30) Foreign Application Priority Data
Jan. 20, 2014 (IL) 230538

(51) Int. Cl. A61B 5/00; G01N 21/3504; A61B 5/083; A61B 5/087; G01N 33/00; G01N 21/59; A61B 5/08; A61B 5/097

(52) U.S. Cl. CPC A61B 5/0059; G01N 21/3504; A61B 5/0836; A61B 5/0873; G01N 33/004; G01N 2201/061; A61B 5/082; A61B 5/097; A61B 5/6819; G01N 2201/022; G01N 21/59

(57) ABSTRACT

A vehicle configured for conveying at least one container. The vehicle has a supporting portion configured for holding said container, and a primary lifting mechanism for controlling elevation of the supporting portion. The lifting mechanism is configured to move the supporting portion between an elevated position and a lowered position. The supporting portion is configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion. The supporting portion in its extended state has projecting areas, projecting in the second outline relative to the first outline and configured for supporting said container.

### TECHNOLOGICAL FIELD

The presently disclosed subject matter relates to systems and methods for storage of containers, and more particularly to storage structures for containers and vehicles for conveying the containers into and from the storage structures.

### BACKGROUND

Systems for storage of containers are used for many years. These systems have various storage structures in which the containers can be accommodated for different periods of time. Storage structures can be located at seaports and harbors, or at any location in which storage of containers is required. The structures can occupy large areas, and for the purpose of reducing these areas, can be structured with elevated floors. With such complex storage structures, conveying containers to and from the storage structures can be a complicated task, which can be carried out by mechanical means and can be facilitated in a variety of techniques.

One type of the above systems includes storage structures in which vehicles (e.g., Automated Guided Vehicles) are used for inserting and extracting the containers to and from the storage structures.

### GENERAL DESCRIPTION

The term ‘vehicle’ refers hereinafter in the specification and the claims to any structure having a chassis and a propulsion arrangement, which is configured for conveying cargo from place to place. For example, the vehicle can be an Automated Guided Vehicle (AGV).

The term ‘container’ refers hereinafter in the specification and the claims to any structure designed for containing material and products for storage or transportation purposes. The container can be an intermodal container (e.g. container, freight container, ISO container, shipping container, hi-cube container, box, conex box and sea can). The container can be a standardized reusable steel box generally used for the safe, efficient and secure storage and movement of materials and products within a global containerized intermodal freight transport system. It is appreciated that the term ‘container’ can also be widely interpreted as an automobile such as a private or a public car, which can be stored by the system of the presently disclosed subject matter.

The term ‘column’ refers hereinafter in the specification and the claims to any structural element of a storage cell of a storage system configured for supporting at least one container thereon.

The phrase ‘base portions’ refers hereinafter in the specification and the claims to any portions of the container that are structured to allow grasping, holding, lifting or supporting the container. The base portions can be located at bottom corner fittings of a container and/or other load transfer area located at the bottom the container. The base portions are defined in various standards, one of which is ISO 1496 titled “Series 1 freight Containers—Specification and testing, part 1”.

According to one aspect of the presently disclosed subject matter there is disclosed a vehicle configured for conveying at least one container. The vehicle has a supporting portion configured for holding said container, and a primary lifting mechanism for controlling elevation of the supporting portion. The lifting mechanism is configured to move the supporting portion between an elevated position and a lowered position. The supporting portion is configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion. The supporting portion in its extended state has projecting areas, projecting in the second outline relative to the first outline and configured for supporting said container.

According to another aspect of the presently disclosed subject matter, above vehicle is used in a system for storage of at least one container.

The above system comprises the following components:
a. a storage structure having a plurality of storage cells, each comprising a plurality of columns with bearing portions configured to support said container within said storage cell;
b. at least one vehicle configured for conveying said container to and from the storage cell. The vehicle has a supporting portion configured for holding said container, and a primary lifting mechanism for controlling elevation of the supporting portion. The lifting mechanism is configured to move the supporting portion between its elevated position and its lowered position. The supporting portion is configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion. The supporting portion in its extended state has projecting areas, projecting in the second outline relative to the first outline and configured for supporting said container.

The ability of the supporting portion to change its dimensions, allows changing the dimensions of the entire vehicle. For example, in the retracted state of the supporting portion combined with the lowered position of the lifting mechanism, the vehicle can have relatively compact dimensions. These dimensions increase the maneuverability of the vehicle within the storage structure, when the vehicle does not carry a container thereon. For instance, a compact vehicle can pass through relatively narrow passages. On the other hand, in the extended state of the supporting portion, the vehicle can have increased dimensioned allowing it to carry a container placed on its projecting areas.

Any one or more of the following features, designs and configurations can be incorporated in any one or more of the aspects of the presently disclosed subject matter, independently or in combinations thereof:

The projecting areas can have outermost points spaced from each other to a distance D1 defining a maximal dimension of the second outline along a first axis of the vehicle. The first outline can have outermost points spaced from each other to a distance D2 defining a maximal dimension along said first axis, which is smaller than D1, allowing the vehicle with its first outline to pass, along a second axis perpendicular to said first axis, between two columns spaced from each other along said first axis to a distance greater than D2 and smaller than D1, without said container thereon.

In a certain embodiment, which will be described in the detailed description, the distance between outermost points of the projecting areas is referred by the reference E1, defining a maximal dimension of the second outline along the first axis of the vehicle, and the outermost points of the first outline is referenced by E2, defining a maximal dimension along said first axis. E1 and E2 respectively correspond with D1 and D2.

On one hand, the dimension D1 is dictated by the location of the base portions of the container so as to correctly support the container during its transportation. On the other hand, the dimension D2 is dictated by the maneuverability requirement of the vehicle within the storage structure (e.g., the distance between different elements of the storage structure, such as columns of storage cells). The ability of the supporting portion to be retracted or extended, allows providing a vehicle in which D2<D1, so that its maneuverability in the lowered position of its lifting mechanism is improved. The columns can comprise lower portions characterized by a minimal length dimension in the first axis associated with the distance therebetween (R1) and the following condition is fulfilled: D2<R1<D1, so that at least in the retracted state of the supporting portion, transportation of the vehicle along said second axis into said storage cell, in the lowered position of the supporting portion without said container thereon is allowed, and in said extended state of the supporting portion, said transportation is prevented.

When the condition D2<R1<D1 is fulfilled, the maneuverability of the vehicle is improved, and the following is allowed:
When the vehicle is characterized by the first outline, the condition D2<R1 is fulfilled, so that the vehicle is able to pass between the column with its lifting mechanism in the lowered position and without the container thereon;
When the vehicle is characterized by the second outline, the condition R1<D2 is fulfilled, so that the vehicle can support a container thereon with its lifting mechanism in the elevated position.

The columns can comprise upper portions characterized by a minimal length dimension in the first axis associated with the distance therebetween (R2) and the following condition is fulfilled: D2<R2<D1, so that at least in the retracted state of the supporting portion, movement of the supporting portion between its elevated position and its lowered position is allowed.

According to specific examples, when the condition D2<R2<D1 is fulfilled, so that at least in the extended state of the supporting portion, movement of the supporting portion between its elevated position and its lowered position can be prevented.

The columns can have straight elongated shape with their bearing portions as horizontal flat surfaces, so that the following condition is fulfilled: R1=R2.

According to the example of straight elongated columns, the peripheral dimensions of the storage cell can be not greater than the peripheral dimensions of a container stored therein, and this allows designing a storage structure in which containers are disposed in proximity to each other. This design can be efficient in space exploitation by the storage structure, and thereby can allow accommodating more containers in a given space.

The upper portions of the columns can protrude into an interior space of their storage cell with respect to the lower portions, so that the following condition is fulfilled: R1>R2.

Each of said projecting areas can be associated with a corresponding retractable element that is retracted in the retracted state of the supporting portion and is extended in the extended state of the supporting portion.

The supporting portion can comprise a plurality of recesses, each configured at least partially to accommodate at least one of said retractable elements in the retracted state of the supporting portion.

The projecting areas can comprise a securing arrangement configured for securing the container to the supporting portion, thereby preventing the container from moving with respect to the supporting portion at least during transportation of the container by the vehicle. The securing arrangement can allow transporting the container by the vehicle in the elevated position of the lifting mechanism. This can eliminate the need for moving the lifting mechanism to its lowered position prior to the transportation of the container, which can save time.

At least part of the projecting areas can comprise a guiding arrangement configured for guiding the container during loading thereof on the vehicle for properly locating the container with respect to the supporting portion. The guiding arrangement can improve the efficient of the container loading operation.

The securing arrangement and the guiding arrangement can be integrated in a common securing-guiding arrangement.

At least one of said securing arrangement and said guiding arrangement can be configured to assume a folded unoperative position and an unfolded operative position.

The supporting portion can have a rectangular shape defined by four corners, and the projecting areas can be disposed at said corners. These projecting areas can be suitable for carrying the container from its bottom corner fittings.

The supporting portion can have a rectangular shape defined by four corners with sides therebetween, and said projecting areas can be disposed at least part of said sides.

The vehicle can comprise an auxiliary arrangement configured for holding the container instead of said supporting portion for allowing the supporting portion to shift between its extended state and its retracted state.

The auxiliary arrangement can be essential when the same base portions are used for supporting the container by the supporting portion and for supporting it by the bearing portions. In this case, the auxiliary arrangement can support the container from alternative base portions other than said base portions, so as to facilitate shifting the container from the supporting portion of the vehicle to the bearing portions of the storage cell.

The auxiliary arrangement can comprise one or more grasping elements and a secondary lifting mechanism. The grasping elements can be configured for assuming a grasping state for grasping the container from alternative base portions other than said base portions, thereby allowing movement of the supporting portion or the container with respect to each other by the secondary lifting mechanism so as to allow shifting of the supporting portion between its extended state and its retracted state, and a folded state associated with the first outline of the vehicle when seen from above.

The auxiliary arrangement can further comprise a middle portion disposed under said supporting portion and between the primary lifting mechanism and the second lifting mechanism, and the grasping elements can be mounted to the middle portion.

The supporting portion can be constituted by a plurality of supporting portions, each associated with a different portion of the vehicle.

The supporting portion and the primary lifting mechanism can be add-ons mountable on a body of a vehicle.

The projecting areas can have outermost points spaced from each other to a distance D1′ (also referred as E1′ in one embodiment which will be described in the detailed description) defining a maximal dimension of the second outline along the second axis of the vehicle, and said first outline can have outermost points spaced from each other to a distance D2′ (also referred as E2′ in one embodiment which will be described in the detailed description) defining a maximal dimension along the second axis, which is smaller than D1′, allowing the vehicle with its first outline to pass, along the first axis perpendicular to said second axis, between columns of a cell spaced from each other along said second axis to a distance greater than D2′ and smaller than D without said container thereon and, when having the second outline, to locate said container on the bearing portions of the columns of said cell. The columns can comprise lower portions characterized by a minimal length dimension in the second axis associated with the distance therebetween (R1′) and the following condition is fulfilled: D2′<R r<D1′, so that at least in the retracted, transportation of the vehicle along said second axis into said storage cell, in the lowered position of the supporting portion without said container thereon is allowed, and in said extended state of the supporting portion, said transportation is prevented.

According to one example, the vehicle can be capable of transporting into and from the storage cell along both first and second axes, at least in the retracted state of the supporting portion and the lowered position of the lifting mechanism.

The columns can comprise upper portions characterized by a minimal length dimension in the second axis associated with the distance therebetween (R2′) and the following condition is fulfilled: D2′<R2′<D1′, so that at least in the retracted state of the supporting portion, movement of the supporting portion between its elevated position and its lowered position is allowed.

The conditions: D2′<R2′<D1′ can be fulfilled, so that at least in the extended state of the supporting portion, movement of the supporting portion between its elevated position and its lowered position is prevented.

According to yet another aspect of the present subject matter there is disclosed a method for using a system for storage of at least one container. The method can comprise steps of:
a. providing said system for storage of at least one container, comprising:
   a storage structure having a plurality of storage cells, each comprising a plurality of columns with bearing portions; and
   at least one vehicle, having a supporting portion and a primary lifting mechanism configured to move the supporting portion between an elevated position and a lowered position. The supporting portion can be configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion. The supporting portion in its extended state has projecting areas, projecting in the second outline relative to the first outline and configured for supporting said container;
b. shifting said supporting portion to its extended state;
c. loading the container on said supporting portion by placing base portions of the container on said projecting areas;
d. transporting the vehicle with the container thereon into the storage cell while the supporting portion is disposed in its elevated position;
e. moving said supporting portion to its lowered position by said primary lifting mechanism, thereby placing the container on said bearing portions;
f. shifting the supporting portion to its retracted state; and
g. transporting the vehicle without the container thereon from the storage cell.

The method can further comprise the steps of steps of:
h. transporting the vehicle to the storage cell while a container is stored in the storage cell and the supporting portion is in its lowered position and its retracted state;
i. shifting the supporting portion to its extended state;
j. moving said supporting portion to its elevated position by said primary lifting mechanism, thereby placing the container on said projecting areas;
k. transposing the vehicle with the container thereon from the storage cell.

According to yet another aspect of the present subject matter there is disclosed a method for using a system for storage of at least one container. The method can comprise steps of:
a. providing a system for storage of at least one container, comprising:
   a storage structure having a plurality of storage cells, each comprising a plurality of columns with bearing portions; and
   at least one vehicle, having a supporting portion, an auxiliary arrangement and a primary lifting mechanism configured to move the supporting portion between an elevated position and a lowered position. The supporting portion can be configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion. The supporting portion in its extended state has projecting areas, projecting in the second outline relative to the first outline and configured for supporting said container. The auxiliary arrangement comprising one or more grasping elements and a secondary lifting mechanism. The grasping elements are configured for assuming a grasping state and a folded state associated with the first outline of the vehicle when seen from above;
b. shifting said supporting portion to its extended state;
c. loading the container on said supporting portion by placing base portions of the container on said projecting areas;
d. transporting the vehicle with the container thereon into the storage cell while the supporting portion is disposed in its elevated position;
e. shifting said grasping elements to their grasping state, thereby grasping the container from alternative base portions other than said base portions;
f. moving of the supporting portion or the container with respect to each other by the secondary lifting mechanism;
g. shifting the supporting portion to its retracted state;
h. moving said supporting portion to its lowered position by said primary lifting mechanism, thereby placing the container on said bearing portions;
i. shifting said grasping elements to their folded state; and
j. transporting the vehicle without the container thereon from the storage cell.

The method can further comprise the steps of:
k. transporting the vehicle to the storage cell while a container is stored in the storage cell and the supporting portion is in its lowered position and its retracted state;
l. shifting said grasping elements to their grasping state, thereby grasping the container from alternative base portions other than said base portions;
m. moving said supporting portion to its elevated position by said primary lifting mechanism;
n. shifting the supporting portion to its extended state;
o. moving of the supporting portion or the container with respect to each other by the secondary lifting mechanism, thereby placing the container on said projecting areas;
p. shifting said grasping elements to their folded state;
q. transposing the vehicle with the container thereon from the storage cell.

According to another aspect of the presently disclosed subject matter there is disclosed a system for storage of at least one container, comprising:
a. a storage structure having a plurality of storage cells, each comprising a plurality of columns with bearing portions configured to support said container within said storage cell;
b. at least one vehicle configured for conveying said container to and from the storage cell, said vehicle having a supporting portion configured for holding said container, and a primary lifting mechanism for controlling elevation of the supporting portion, said lifting mechanism being configured to move the supporting portion between its elevated position and its lowered position;
c. a guiding-securing arrangement mechanically associated with said supporting portion and configured for: guiding the container during loading thereof on the vehicle for properly locating the container with respect to the supporting portion, and securing the container to the supporting portion so as to prevent the container from moving with respect thereto at least during transportation of the container by the vehicle.

Having the securing arrangement mechanically associated with the supporting portion can allow transporting the container by the vehicle in the elevated position of the lifting mechanism. This can eliminate the need for moving the lifting mechanism to its lowered position prior to the transportation of the container, which can save time. In addition, the securing arrangement can reduce risk of movement of the container with respect to the vehicle during transportation.

The guiding arrangement can improve the efficiency of the container loading operation and to facilitate proper location of the container on the vehicle.

The supporting portion can comprise supporting areas configured for supporting said container at base portions thereof, and wherein said supporting portion is structured so that when the container is received thereon, additional base portions of the container are exposed, when seen from below, for being placed on said bearing portions when the supporting portion is moved from its elevated position to its lowered position.

The vehicle can have a first axis and a second axis perpendicular thereto and the supporting portion can have a first maximal length dimension (L1) in said first axis. The columns can comprise lower portions characterized by a minimal length dimension in the first axis associated with the distance therebetween (R1) and the following condition can be fulfilled: R1>L1, so that transportation of the vehicle along said second axis into said storage cell, at least in the lowered position of the supporting portion without said container thereon is allowed.

The columns can comprise upper portions characterized by a minimal length dimension in the second axis associated with the distance therebetween (R2) and said upper portions can protrude into an interior space of their storage cell with respect to the lower portions, so that the following condition is fulfilled: R1>R2.

The supporting portion can have a first maximal length dimension (L1′) in said 20 second axis, and the columns can comprise lower portions characterized by a minimal length dimension in the second axis associated with the distance therebetween (R1′). the following condition can be fulfilled: R1′>L1′, so that transportation of the vehicle along said first axis into said storage cell, in the lowered position of the supporting portion without said container thereon is allowed.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic front view of a storage system, in accordance with one example of the presently disclosed subject matter;

FIG. 2 is Figure B1 of the ISO 1496 standard, showing an example of a bottom surface of a container which can be stored within the system of FIG. 1;

FIG. 3A is a perspective side view of a vehicle, in accordance with one example of the presently disclosed subject matter;

FIG. 3B is a perspective side view of the vehicle of FIG. 3A, with a container disposed thereon;

FIG. 3C is a top view of the vehicle of FIG. 3A, in a retracted state of its supporting portion;

FIG. 3D is a top view of the vehicle of FIG. 3A, in an extended state of its supporting portion;

FIG. 3E is an enlarged view of a retractable element of the vehicle of FIG. 3B in its unfolded operative position;

FIG. 4A is a front view of the vehicle of FIG. 3A, in a retracted state and a lowered position of its supporting portion, located within a storage cell and a container disposed therein;

FIG. 4B is a front view of the vehicle of FIG. 3A, in an extended state and a lowered position of its supporting portion, located within a storage cell and a container disposed therein;

FIG. 4C is a side view of the vehicle of FIG. 3A, in a retracted state and a lowered position of its supporting portion, located within a storage cell and a container disposed therein;

FIG. 4D is a side view of the vehicle of FIG. 3A, in an extended state and a lowered position of its supporting portion, located within a storage cell and a container disposed therein;

FIG. 5A is a perspective view of the vehicle of FIG. 3A, in a retracted state and an elevated position of its supporting portion;

FIG. 5B is a perspective view of the vehicle of FIG. 3A, in an extended state and an elevated position of its supporting portion;

FIG. 5C is a perspective view of the vehicle of FIG. 3A, in an extended state and an elevated position of its supporting portion, and a container loaded thereon;

FIG. 5D is a perspective view of the vehicle of FIG. 5C, with a container disposed thereon;

FIG. 5E is a perspective view of the vehicle of FIG. 5D within a storage cell;

FIG. 5F is a perspective view of the vehicle of FIG. 3A within a storage cell, in an extended state and a lowered position of its supporting portion, with a container supported by the storage cell;

FIG. 5G is a perspective view of the vehicle of FIG. 3A within a storage cell, in a retracted state and a lowered position of its supporting portion, with a container supported by the storage cell;

FIG. 5H is a perspective view of a storage cell with a container supported thereon, and without the vehicle;

FIG. 6A is a side view of a vehicle, in accordance with another example of the presently disclosed subject matter;

FIG. 6B is a side view of the vehicle of FIG. 6A, with a container disposed thereon;

FIG. 6C is a top view of the vehicle of FIG. 6A, in a retracted state of its supporting portion;

FIG. 6D is a top view of the vehicle of FIG. 6A, in an extended state of its supporting portion;

FIG. 6E is an enlarged view of a bottom corner fitting of a container to be transported by the vehicle of FIG. 6A;

FIG. 7A is a front view of the vehicle of FIG. 6A within a storage cell, in a retracted state and a lowered position of its supporting portion;

FIG. 7B is a front view of the vehicle of FIG. 6A within illustrative columns of a storage cell, in an extended state and a lowered position of its supporting portion;

FIG. 7C is a side view of the vehicle of FIG. 6A within a storage cell, in a retracted state and a lowered position of its supporting portion;

FIG. 7D is a side view of the vehicle of FIG. 6A within illustrative columns a storage cell, in an extended state and a lowered position of its supporting portion;

FIG. 8A is a front view of the vehicle of FIG. 6A within a storage cell, in a retracted state and a lowered position of its supporting portion;

FIG. 8B is a front view of the vehicle of FIG. 6A within a storage cell, in an extended state and an elevated position of its supporting portion;

FIG. 8C is a side view of the vehicle of FIG. 6A within a storage cell, in a retracted state and a lowered position of its supporting portion;

FIG. 8D is a side view of the vehicle of FIG. 6A within a storage cell, in an extended state and an elevated position of its supporting portion;

FIG. 9A is a side view of the vehicle of FIG. 6A, in the extended state and the elevated position of its supporting portion;

FIG. 9B is a side view of the vehicle of FIG. 6A, in the extended state and the elevated position of its supporting portion, with a container disposed thereon;

FIG. 9C is a side view of the vehicle of FIG. 6A, in the extended state and the elevated position of its supporting portion, with a container disposed thereon, inside of a storage cell;

FIG. 9D is a side view of the vehicle of FIG. 6A, in the extended state and the elevated position of its supporting portion and in a grasping state of its grasping elements with a container disposed thereon, inside of a storage cell;

FIG. 9E is a side view of the vehicle of FIG. 6A, in the extended state and the elevated position of its supporting portion and in a grasping state of its grasping elements with a container disposed thereon, inside of a storage cell;

FIG. 9F is a side view of the vehicle of FIG. 6A, in the retracted state and the elevated position of its supporting portion and in the grasping state of its grasping elements with a container disposed thereon, inside of a storage cell;

FIG. 9G is a side view of the vehicle of FIG. 6A, in the retracted state and the lowered position of its supporting portion and in the grasping state of its grasping elements with a container disposed thereon, inside of a storage cell;

FIG. 9H is a side view of the vehicle of FIG. 6A, in the retracted state and the lowered position of its supporting portion and in a folded state of its grasping elements, inside of a storage cell, with a container supported by the storage cell;

FIG. 10A is a perspective side view of a vehicle, in accordance with another example of the presently disclosed subject matter;

FIG. 10B is a perspective side view of the vehicle of FIG. 10A, with a container disposed thereon;

FIG. 10C is a top view of the vehicle of FIG. 10A, inside a storage cell, without a container;

FIG. 11 is an enlarged view of a guiding-securing projection of the vehicle of FIG. 10A;

FIG. 12 is a perspective side-bottom view of the vehicle of FIG. 10A, in an elevated position of its supporting portion, with a container disposed thereon, within a storage cell;

FIG. 13A is a front view of the vehicle of FIG. 10A within a storage cell, in the elevated position of its supporting portion, with a container disposed thereon;

FIG. 13B is a side view of the vehicle of FIG. 10A within a storage cell, in the elevated position of its supporting portion, with a container disposed thereon;

FIG. 13C is a front view of the vehicle of FIG. 10A within a storage cell, in the lowered position of its supporting portion, with a container supported by the storage cell;

FIG. 13D is a side view of the vehicle of FIG. 10A within a storage cell, in the lowered position of its supporting portion, with a container supported by the storage cell;

### DETAILED DESCRIPTION OF EMBODIMENTS

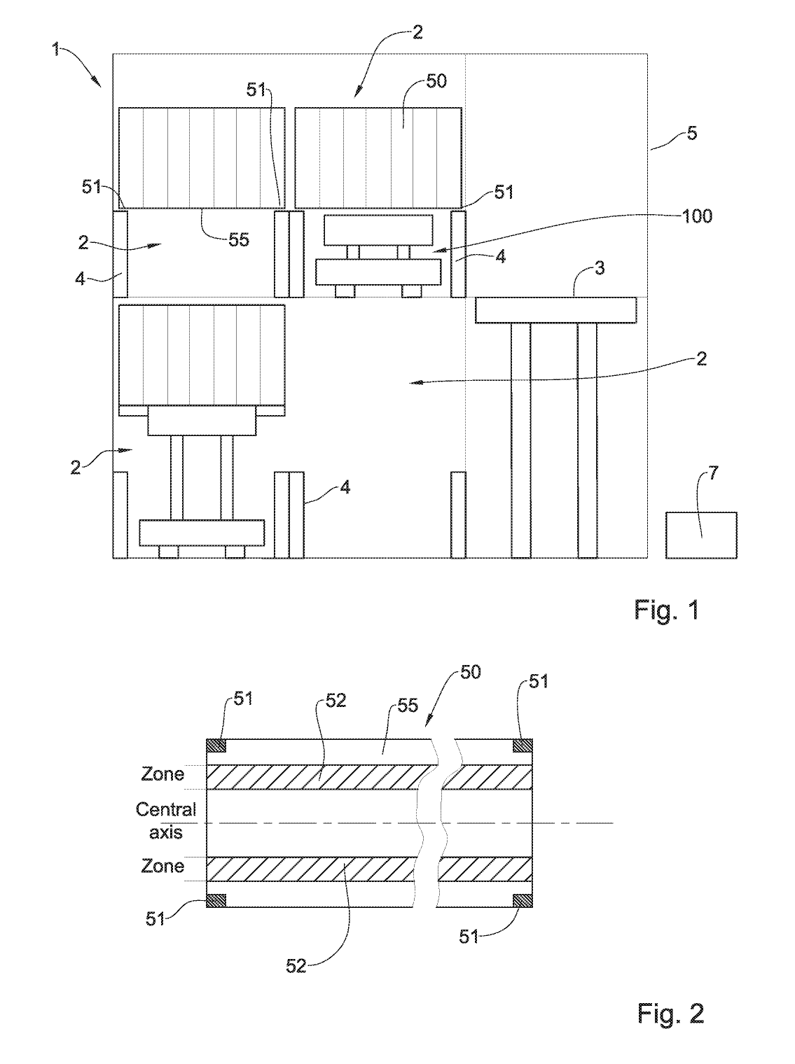

Attention is first directed to FIG. 1 of the drawings illustrating a storage system generally designated 1, for storage of containers, in accordance with one example of the presently disclosed subject matter. The illustrated storage system 1 comprises a storage structure 5 constructed of a plurality of storage cells 2. Each storage cell 2 is defined by four corner columns 4 for supporting a container 50 while being stored therein. The storage system 1 is configured with two floors: an upper floor and a lower floor, each of which comprises two storage cells 2. Access to the upper floor is provided by an elevator 3. The storage system 1 further comprises vehicles 100 for conveying the containers into and from the storage cells 2. The vehicles are Autonomous Guided Vehicles (AGVs) that are wirelessly controllable by a controlling unit 7. A detailed explanation regarding the structure of the vehicles 100 is provided below.

Reference is now made to FIG. 2, for explaining the structure of the bottom portion 55 (shown in FIGS. 1 and 2) of the container 50. The bottom portion 55 has base portions which are structured so as to allow grasping, holding, lifting or supporting the container 50 by external means.

The base portions are defined in various standards, one of which is ISO 1496 titled “Series 1 freight Containers—Specification and testing, part 1”. The ISO 1496 standard defines the base portions as follows:

“All containers shall be capable of being supported by their bottom corner fittings only . . .

. . . All containers, other than 1 D and 1 DX, shall also be capable of being supported only by load transfer areas in their base structure. Consequently, these containers shall have end transverse members and sufficient intermediate load transfer areas (or a flat underside) of sufficient strength to permit vertical load transfer to or from the longitudinal member of a carrying vehicle. Such longitudinal members are assumed to lie within the two 250 mm wide zones defined by the broken lines in figure B1”.

According to the above definition, the base portions can be related to two types of portions: bottom corner fittings 51 and load transfer areas 52. In particular, the bottom corner fittings 51 allow carrying therefrom all types of containers, and the load transfer areas 52 allow carrying therefrom all types of container, other than the 1D and the 1DX types.

Referring back to FIG. 1, the container 50 is not related to the 1D and the 1DX types, and therefore, it can be carried by both by the bottom corner fittings 51 and by the load transfer areas 52. As shown in FIG. 1, the bottom corner fittings 51 are located at corners of the bottom portion 55 and are configured to allow the container 50 to be supported by the columns 4 of the storage cell 2 while being stored therein. The load transfer areas 52 are disposed along the bottom portion 55 and are configured to allow the container 50 to be supported by the vehicle 100 during its transportation.

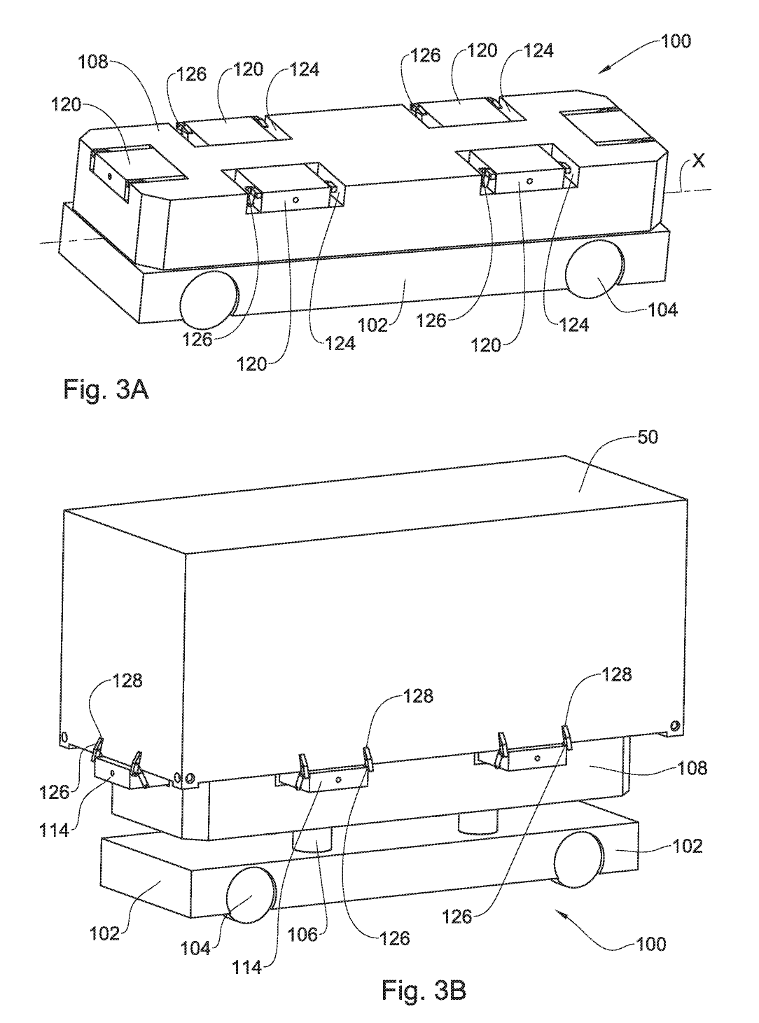

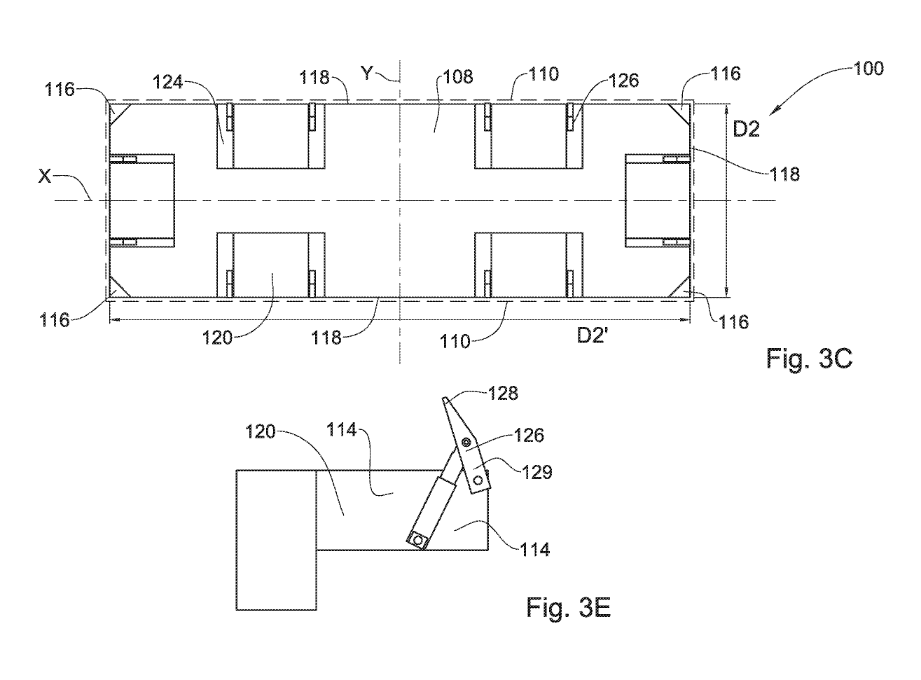

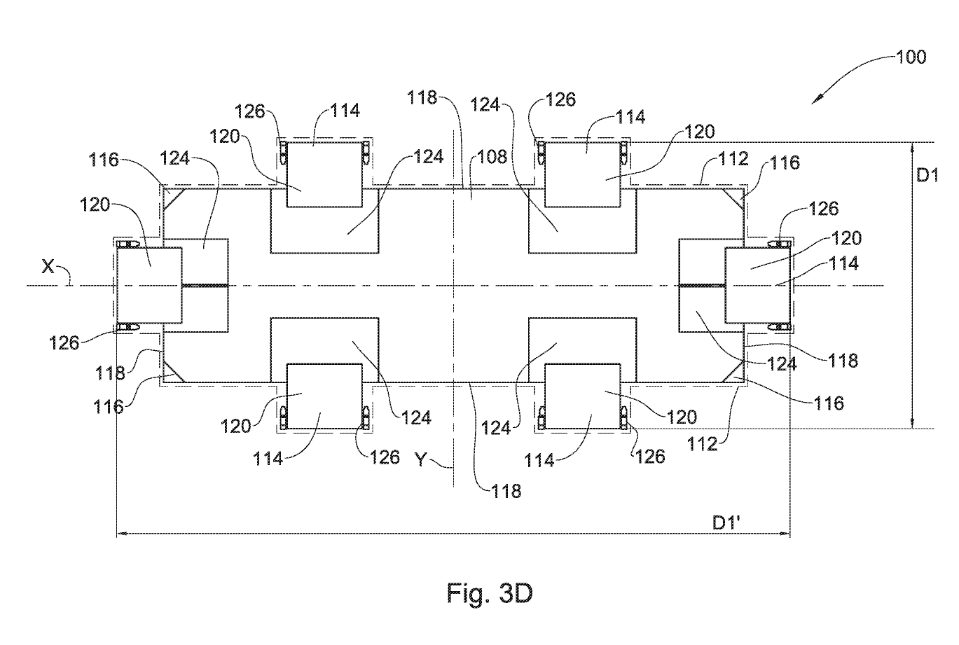

Reference in now made to FIGS. 3A to 3E, 4A to 4D and 5A to 5H, in which the vehicle 100 is shown in a detailed manner.

The vehicle 100 is characterized by changeable dimensions, which on one hand allow it to maneuver within the storage system 1 between the columns 4, and on the other hand, to carry a container when needed.

The maneuverability of the vehicle 100 within the storage system 1 is a function of the dimensions of the vehicle 100 at present moment, as well as the distance between the columns 4 forming the storage cells 2. In order to be able to pass between the columns 4, the vehicle 100 should have reduced dimensions.

In order to convey the container 50 to and from the storage cell 2, the vehicle 100 should have increased dimensions, which allow it support the container at the load transfer areas 52, and should also allow placing its bottom corner fittings 51 on the columns 4.

The vehicle 100 is an AGV that comprises: a body 102, a propulsion arrangement (not shown), omnidirectional wheels 104 mechanically associated with the propulsion arrangement, and a computer (not shown) that controls the operation of the propulsion arrangement. The omnidirectional wheels 104 allow the vehicle 100 to move along a first axis, namely Y axis (shown in FIGS. 3C and 3D), as well as a second axis, namely X axis, which is perpendicular to the Y axis. A primary lifting mechanism 106, in the form of four pistons, is mounted on the body 102 and is configured for controlling elevation of a supporting portion 108 between a lowered position, shown in FIG. 3A and an elevated position, shown in FIG. 3B. The primary lifting mechanism 106 is operative for allowing insertion and extraction of the container 50 to and from the storage cell 2 in an elevated position, in which the container is located above the columns 4.

The supporting portion 108 is configured for holding the container 50 thereon, and configurable between a retracted state associated with a first outline 110 of the vehicle when seen from above (marked in dashed line in FIG. 3C), and an extended state associated with a second outline 112 of the vehicle when seen from above (marked in dashed line in FIG. 3D).

The supporting portion 108 comprises retractable elements 120 horizontally movable with respect to the supporting portion 108, so that they are retracted in the retracted state of the supporting portion 18, and extended in the extended state of the supporting portion 18.

In the extended state of the supporting portion 108, projecting areas 114 project in the second outline 112 relative to the first outline 110 and are configured for supporting the container 50. Each one of the projecting areas 114 is associated with a corresponding retractable element 120, while being retracted.

Each of the retractable elements 120 is retractable into a respective recess 124 formed within the supporting portion 108. As can be seen in FIG. 3C, in the retracted state of the supporting portion 108, the retractable elements 120 are fully accommodated within the recesses 124. Moreover, as can be seen in FIG. 3D, the retractable elements 120 are only partially accommodated within recesses 124, while mainly projecting out of the first outline of the vehicle, forming the respective projecting areas 114.

As shown in FIG. 3A, the supporting portion 108 is in its lowered position and retracted state. In these position and state, the vehicle 100 is in its most compact form, and its can freely travel within the storage structure 5 while passing between the columns 4 of the storage cells 2 (including those in which containers are stored) and without the container 50 thereon.

As shown in the drawings, the vehicle 100 of FIGS. 3A and 3C has the first outline 110. The first outline 110 is characterized by outermost points spaced from each other to a distance D2, defining a maximal dimension along the Y axis. The first outline 110 is also characterized by outermost points spaced from each other to a distance D2′, defining a maximal dimension along the X axis.

As shown in FIG. 3B, the supporting portion 108 is in its elevated position and extended state. In these position and state, the dimensional of the vehicle 100 are increased to as to carry the container 50 thereon.

As shown in the drawings, the vehicle 100 of FIGS. 3B and 3D has the second outline 112. The second outline 112 is characterized by outermost points spaced from each other to a distance D1, defining a maximal dimension along the Y axis. The second outline 112 is also characterized by outermost points spaced from each other to a distance D1′, defining a maximal dimension along the X axis.

The dimensions D1 and D1′ are dictated by the location of the base portions of the container 50, i.e., the load transfer areas 52, and the distance therebetween, so as to correctly support the container 50 during its transportation. In the illustrated example, the supporting portion 108 has a rectangular shape defined by four corners 116 with sides 118 therebetween, and the projecting areas 114 project from the sides 118.

As shown in FIG. 3B, the retractable elements 120 in their retracted form, support the container 50 from its load transfer areas 52, and not from the bottom corner fittings 51. Therefore, the vehicle 100 is configured for carrying containers related to types other than 1D and 1DX. The retractable elements 120 comprise a guiding-securing arrangement in the form of guiding-securing lugs 126, best seen in FIGS. 3B and 3E. The guiding-securing lugs 126 are configured to assume a folded unoperative position, seen in FIG. 3A, and an unfolded operative position seen in FIG. 3B. An enlarged view of a guiding-securing lug 126 in its unfolded operative position is shown in FIG. 3E.

When disposed in its unfolded operative position, each of the guiding-securing lugs 126 is protruding upwardly from a respective retractable element 120. In this position, the guiding-securing lugs 126 are configured to guide the container 50 during loading thereof on the vehicle 100 for properly locating it with respect to the supporting portion 108. The effect of guiding is gained by the protrusion of the guiding-securing lugs 126. During the loading of the container 50 on the supporting portion 108, the operator in charge of the loading can locate the container between distal ends 128 of the protruding guiding-securing lugs 126, which are distal with respect to the retractable elements 120, and then slide the container downwardly along the guiding-securing lugs 126 to the proper location on the supporting portion 108. The location of the proximate end 129 on the retractable elements 120 facilitates proper location of the container 50 on the supporting portion 108.

In addition, the guiding-securing lugs 126 constitute securing elements, preventing unintentional movement of the container 50 with respect to the supporting portion 108. The securing effect is gained by the protrusion of the guiding-securing lugs 126, which act as stopping elements, preventing the container from moving horizontally. As best seen in FIG. 3B, the guiding-securing lugs 126 surround the container and stopping it from moving. This arrangement facilitates transportation of the vehicle 100 with the container 50 thereon, in both the elevated and lowered positions of the supporting portion 108.

The folded unoperative position of the guiding-securing arrangement lugs 126 is provided for enabling a compact form of the supporting portion, and is combinable at least with the retracted state of the supporting portion 108, as illustrated in FIG. 3A.

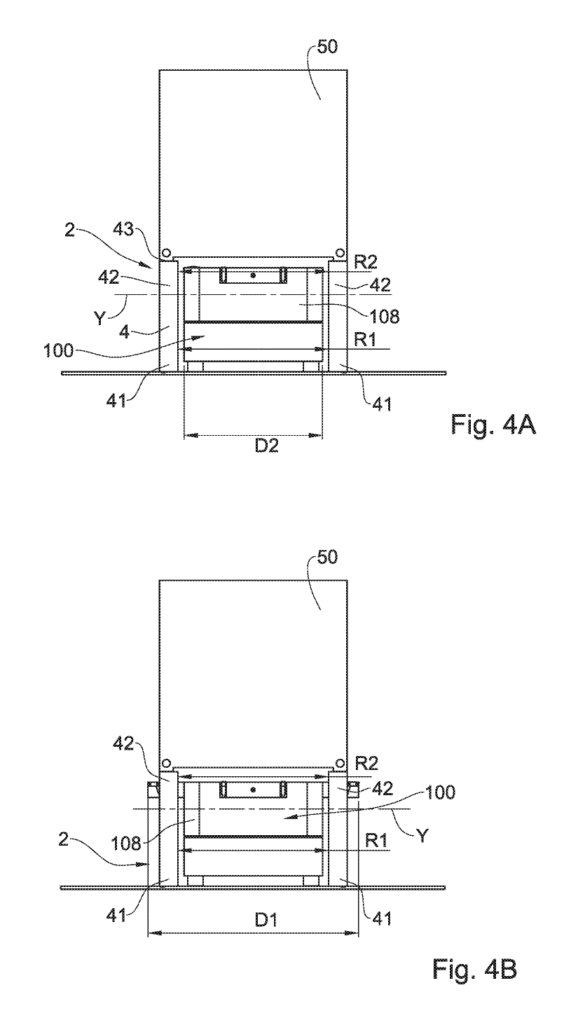

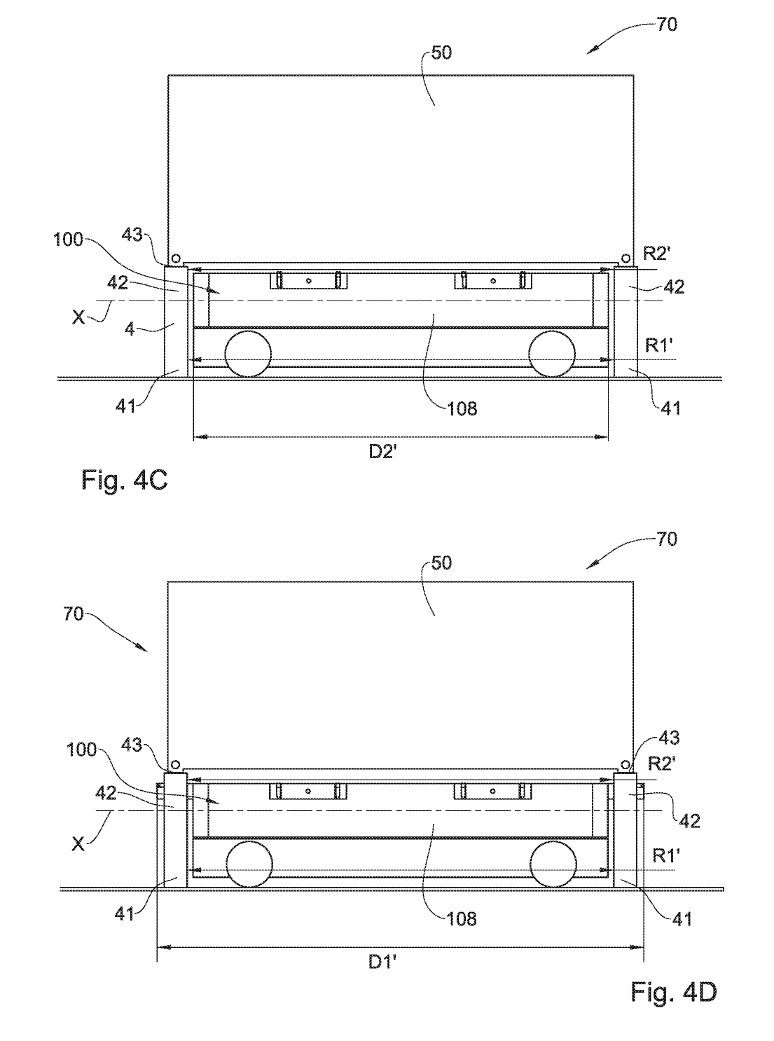

Reference is now made to FIGS. 4A to 4D in order to explain in a detailed manner the relationship between the vehicle 100 and the storage cell 2, and in particular the compatibility of their dimensions to each other.

As mentioned above, the dimensions of vehicle 100 are configured so as to allow the vehicle 100 to pass between the columns 4 of the storage cell 2, along both X and Y axes, in the retracted state and the lowered position of the supporting portion 108.

FIGS. 4A and 4B illustrate how the increase in dimensions of the vehicle 100 along axis X from the first to the second outline influences on the vehicle's ability to pass between the columns 4 along axis X.

The distance between the columns 4 along Y axis is greater than D2 and smaller than D1 and thereby allowing the vehicle 100 with its first outline 110 (which is associated with the retracted state of the supporting portion 108 and with the distance D2) to pass therebetween along X axis, on one hand, and preventing its passage therebetween when the vehicle 100 is with its second outline 112 (which is associated with the extended state of the supporting portion 108 and with the distance D1).

The columns 4 have lower portions 41 characterized by a minimal length dimension therebetween R1 in the first axis, i.e. Y axis in the drawings, and the following condition is fulfilled: D2<R1<D1. The distance R1 is related to the dimensions of the vehicle 100 in the lowered position of the supporting portion 108, so that this condition assures that in the lowered position and the retracted state of the supporting portion 108, the vehicle 100 can pass between the columns when moving along the X axis. It can be easily seen from FIG. 4B that in the lowered position and the extended state of the supporting portion 108, the vehicle 100 cannot pass between the columns 4 when moving along the X axis.

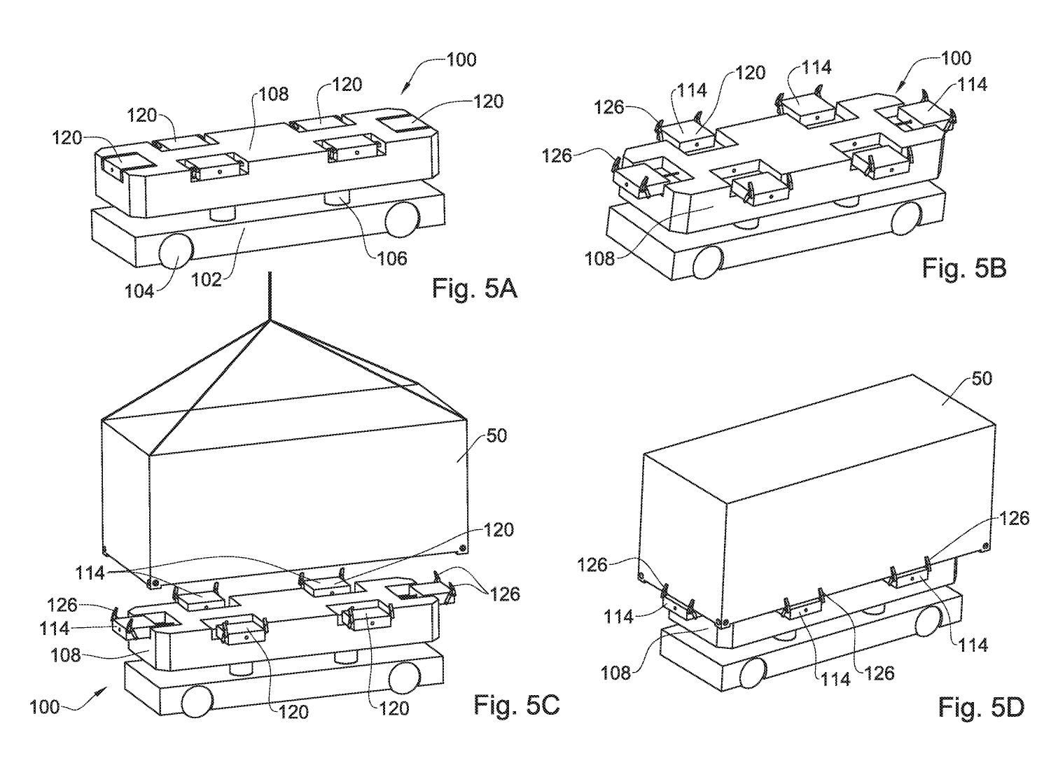

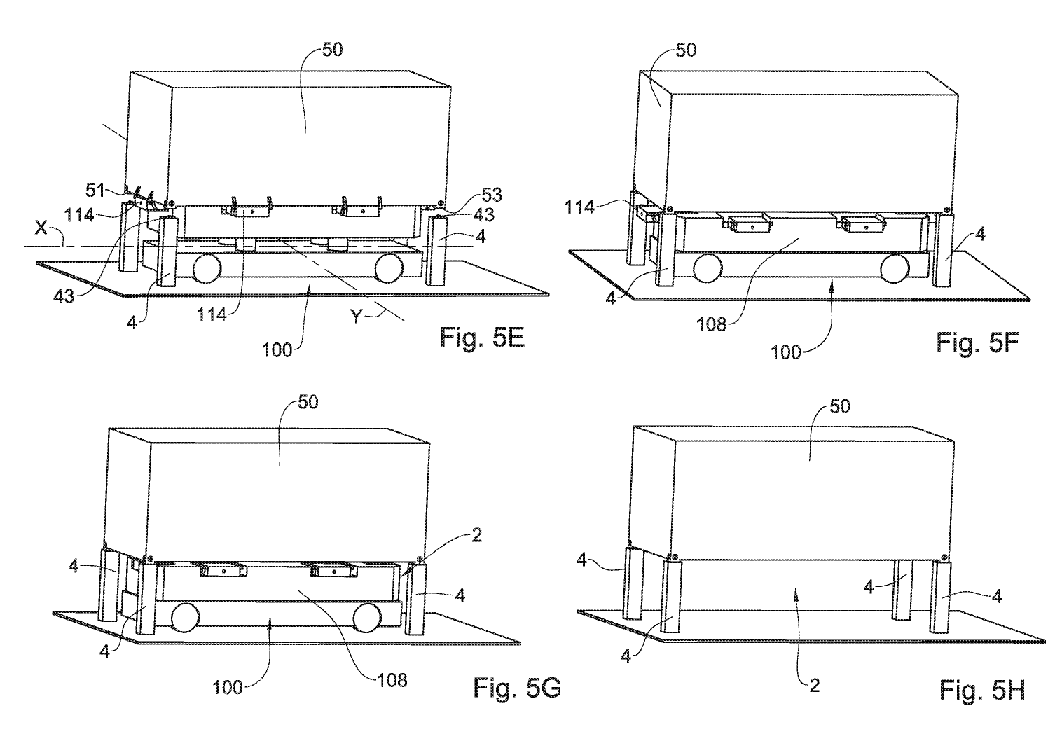

The columns 4 also have upper portions 42 characterized by a minimal length dimension R2 therebetween in the Y axis. The distance R2 is related to a distance between bearing portions 43 (seen best in FIG. 5E) of the columns 4. Bearing portions 43 are horizontal flat surfaces, located at upper portions 42 of the columns 4, and are configured for supporting the container 50 thereon, while being stored at the storage cell 2. The location and the dimensions of the bearing portions 43 are dictated by the location and the dimensions of the base portions of the container 50, so as to properly support to container 50 within the storage cell 2.

The columns 4 are characterized by a straight elongated shape, so that the following condition is fulfilled: R1=R2. In addition, the peripheral dimensions of the storage cell 2 are substantially equal to the peripheral dimensions of the container 50.

As shown in FIGS. 4C and 4D, the lower portions 41 are characterized by a minimal length dimension therebetween R1′, and the upper portions 42 are characterized by a minimal length dimension R2′, both in the X axis. The relationship between the parameters D1′, D2′, R1′ and R2′ is similar in a respective manner to the relationship between the parameters D1, D2, R1 and R2.

As can be seen from FIGS. 4A to 4D, the dimensions of the vehicle 100, the container 50 and the columns 4, allow designing a storage system in which containers 50 are disposed in proximity to each other, as can be seen in FIG. 1. This allows saving storage space, and increasing the efficiency of exploiting a given storage space.

Reference is now made to FIGS. 5A to 5H, illustration the steps of the method of inserting the container 50 into the storage cell 2.

In FIG. 5A, the vehicle 100 is shown with the supporting portion 108 in its elevated position and retracted state. For being able to properly receive the container 50 thereon, the supporting portion 108 is shifted to its extended state, as seen in FIG. 5B. It can also be seen in FIG. 5B that the guiding securing lugs 126 are shifted to their unfolded operative position.

FIGS. 5C and 5D illustrate the process of loading the container 50 on the supporting portion 108, by placing the base portions of the container 50 on the retractable elements 120, and in particular, on the projecting areas 114. Locating the container 50 in its proper location on the supporting portion 108 is facilitated by the guiding of the guiding-securing lugs 126. After loading the container 50 onto the supporting portion 108, the container 50 is securely held thereon by the guiding-securing lugs 126, which now facilitate a securing arrangement, preventing unintentional movement of the container 50 with respect to the supporting portion 108. With the container 50 secured to the supporting portion 108 by the guiding-securing lugs 26, the vehicle 100 can transport the container into the storage cell 2.

While transportation of the container 50 can be performed in both the lowered and the elevated positions of the supporting portion 108, entrance into the storage cell 2 is possible only with the supporting portion 108 in its elevated position, as best seen in FIG. 5E. In the elevated position, the projecting areas 114 are elevated higher than the bearing portions 43, located at the upper portions 42 of the columns 4. While entering into the storage cell 2, the projecting areas 114, with the container 50 supported thereon, pass above the bearing portions 43 of the columns 40.

Apart from the projecting areas 114, which project from the first outline 110 of the vehicle, other parts of the vehicle 100 do not project from the first outline, i.e. the outermost points are spaced from each other to the distances D2 and D2′. As a result of that, passage of the vehicle 100 between the lower portions 41 of the columns 4, which are spaced from each other in a distance R1 along the Y axis and R1′ along X axis, is facilitated.

The vehicle 100 takes such a position in the cell 2 wherein the bearing portions 43 are located underneath the bottom corner fittings 51 of the container 50, from which the container is to be supported by the columns 2.

The next step involves moving the supporting portion 108 to its lowered position by the primary lifting mechanism 106, thereby placing the bottom corner fittings 51 of the container on the bearing portions 43 of the columns, as seen in FIG. 5F.

Now, with the container 50 being supported by the bearing portions 43 of the columns 4, the vehicle 100 can exit the storage cell 2 without the container 50. Before exiting the storage cell 2, the supporting portion 108 is shifted to its retracted state, thereby decreasing the outline of the vehicle to the first outline 110, with which passage of the vehicle 100 between the columns 4, in the lowered position of the supporting portion 108, is allowed (FIG. 5G). Similarly to the entrance into the storage cell 2, the vehicle 100 can also exit the storage cell 2 from all four directions, i.e. along both axes X and Y.

FIG. 5H shows the storage cell 2 with the container 50 stored therein, after the vehicle 100 has left the storage cell 2.

The method can further comprise steps of taking the container 50 out from a storage cell 2. These steps are reverse steps to the above steps of inserting the container 50 into the storage cell 2. These reverse steps include: transporting the vehicle 100 into the storage cell 2, while the container 50 is stored therein and the supporting portion 108 is in its lowered position and its retracted state (FIG. 5G); shifting the supporting portion 108 into its extended state (FIG. 5F); moving the supporting portion 108 to its elevated position by the primary lifting mechanism 106, thereby placing the container 50 on the projecting areas 114 of the supporting portion 108 (FIG. 5E); and transposing the vehicle 100 with the container 50 thereon from the storage cell 2 (FIG. 5D).

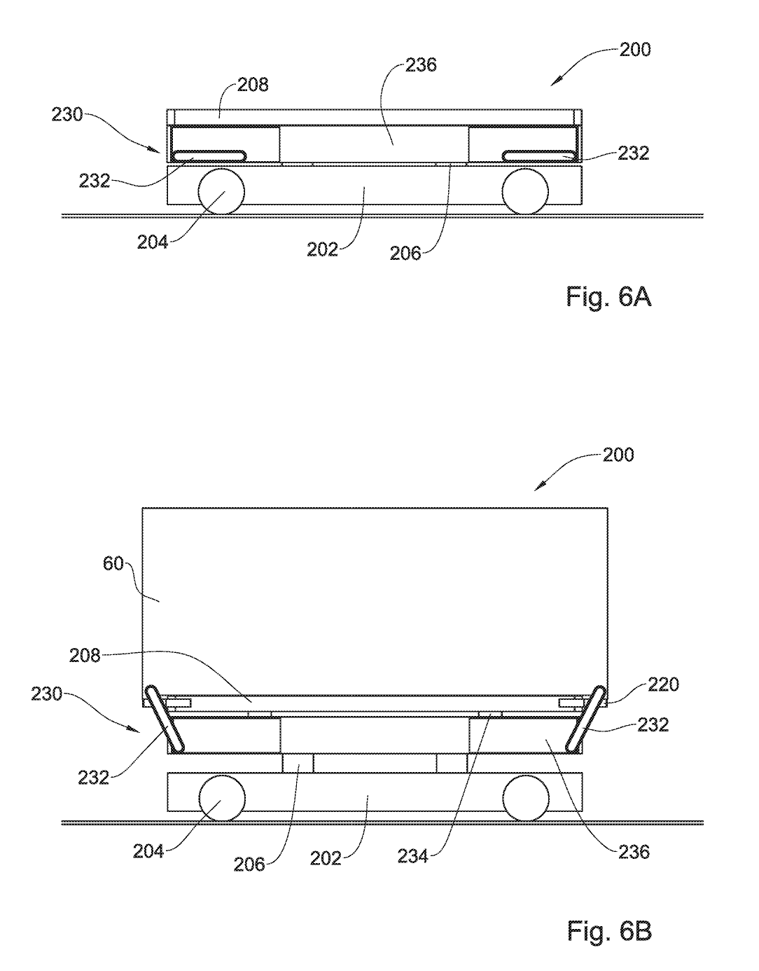

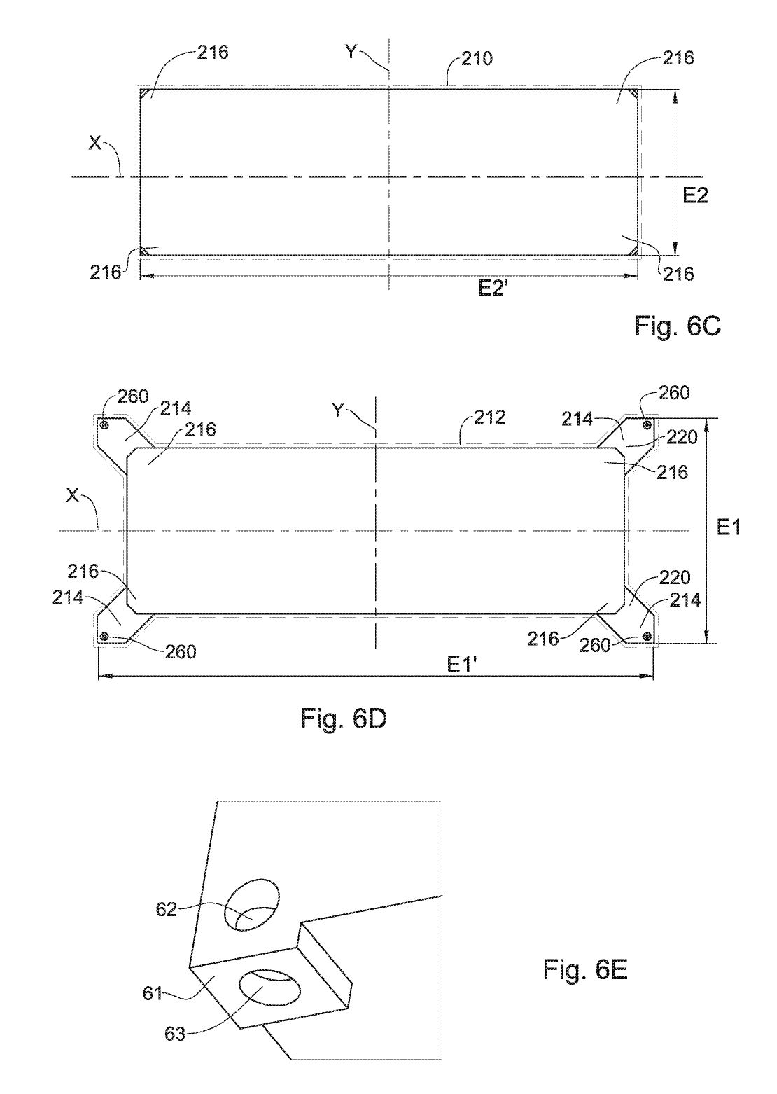

Another example of a vehicle according to the presently disclosed subject matter is vehicle 200, illustrated in FIGS. 6A to 6E, 7A to 7D, 8A to 8D and 9A to 9H.

The vehicle 200 is an AGV that comprises: a body 202, a propulsion arrangement (not shown), omnidirectional wheels 204 mechanically associated with the propultion arrangement, and a computed (not shown) that controls the operation of the propultion arrangement. The omnidirectional wheels 204 allow the vehicle 200 to move along a first axis, namely Y axis (shown in FIGS. 6C and 6D), as well as a second axis, namely X axis, which is perpendicular to the Y axis. A primary lifting mechanism 206, in the form of four pistons, is mounted on the body 202 and is configured for controlling elevation of a supporting portion 208 between a lowered position, shown in FIG. 6A and an elevated position, shown in FIG. 6B. The primary lifting mechanism 206 is operative for allowing insertion and extraction of a container 60 to and from the storage cell 2 in an elevated position, in which the container is located above the columns 4.

The supporting portion 208 is configured for holding the container 50 thereon, and configurable between a retracted state associated with a first outline 210 of the vehicle 200 when seen from above (marked in dashed line in FIG. 6C), and an extended state associated with a second outline 212 of the vehicle 200 when seen from above (marked in dashed line in FIG. 6D).

As shown in FIG. 6A, the supporting portion 208 is in its lowered position and retracted state. In these position and state, the vehicle 200 is in its most compact form, and its can freely travel within the storage structure 5 while passing between the columns 4 of the storage cells 2 (including those in which containers are stored) and without the container 60 thereon.

In the retracted state supporting portion 208, the vehicle 200 is characterized by a first outline 210 (marked in dashed line in FIG. 6C), and has outermost points spaced from each other to a distance E2, defining a maximal dimension along a first axis, i.e. Y axis in the drawings. In this state, the vehicle 200 also has outermost points spaced from each other to a distance E2′, defining a maximal dimension along a second axis, i.e. X axis in the drawings.

As shown in FIG. 6B, the supporting portion 208 is in its elevated position and extended state. As shown in FIG. 6C, in the extended state of the supporting portion 208, projecting areas 214 project in a second outline 212 relative to the first outline 210 and configured for supporting the container 60. The projecting areas 214 have outermost points spaced from each other to a distance E1, seen in FIG. 6D, defining a maximal dimension of the second outline 212 along Y axis. Similarly, the projecting areas 214 have outermost points spaced from each other to a distance E1′, also seen in FIG. 6D, defining a maximal dimension of the second outline 212 along X axis. The dimensions E1 and E1′ are dictated by the location of the base portions of the container 60 so as to correctly support the container 60 during its transportation. The supporting portion 208 has a rectangular shape defined by four corners 216, and the projecting areas 214 are disposed at the corners 216. These projecting areas 214 are suitable for carrying the container from bottom corner fittings 61 thereof, best seen in FIG. 6E, which are similar to bottom corner fittings 51 of container 50.

As can be understood from the drawings, the projecting areas 214 of the vehicle 200 are configured for supporting the container 60 from its bottom corner fittings 61, and not from the load transfer areas, and therefore the vehicle 200 is suitable for carrying all kinds of containers, including the 1D and 1DX type containers.

Each one of the projecting areas 214 is associated with a corresponding retractable element 220. The retractable elements 220 are retracted in the retracted state of the supporting portion 208, and are extended in the extended state thereof. Each one of the retractable elements 220 is retractable into a respective recess (not shown in the drawings) of the supporting portion 208.

The retractable elements 220 comprise a securing arrangement in the form of securing lugs 260, best seen in FIG. 6D.

Each securing lug 260 is protruding upwardly from a respective retractable element 220. The securing lugs 260 constitute securing elements, preventing unintentional movement of the container 60 with respect to the supporting portion 208. The securing effect is gained by the protrusion of the securing lugs 260 into corresponding recesses 63 (seen best in FIG. 6E) in the bottom corner fittings 61, and thereby preventing the container from moving horizontally. This arrangement facilitates transportation of the vehicle 200 with the container 60 thereon, in both the elevated and lowered positions of the supporting portion 208.

Vehicle 200 is adapted to operate in conjunction with the storage cell 2 for storing the container 60 therein.

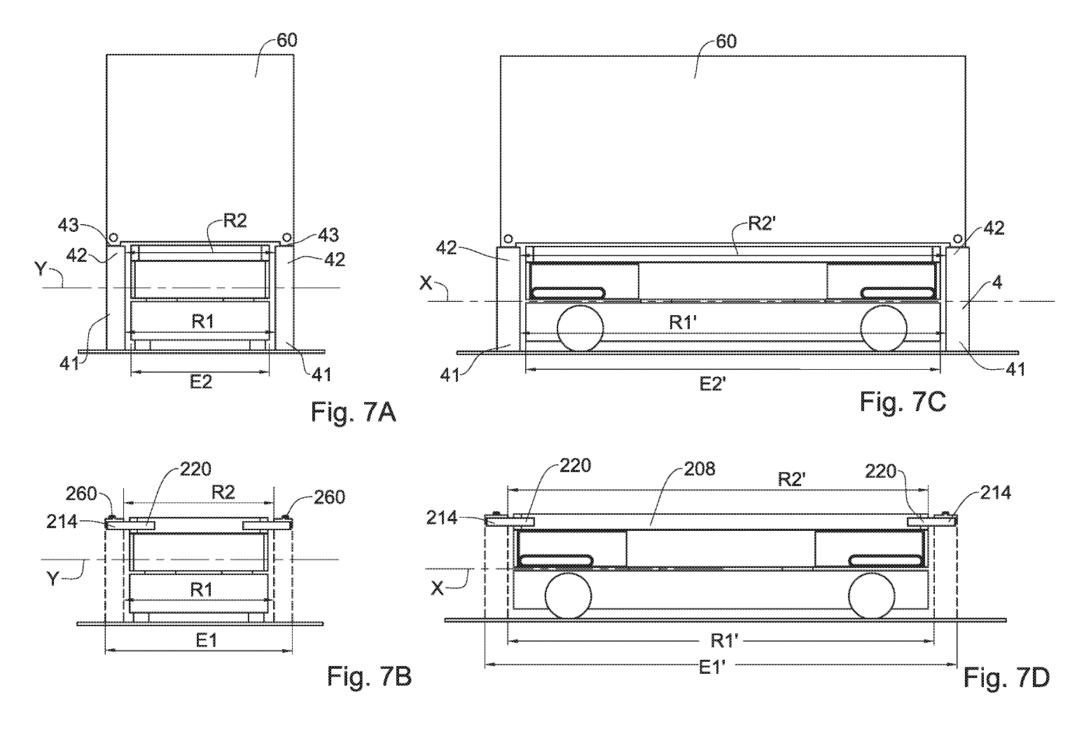

As mentioned above, the dimensions of vehicle 200 are configured so as to allow the vehicle 200 to pass between the columns 4 of the storage cell 2, along both X and Y axes, in the retracted state and the lowered position of the supporting portion 208. FIGS. 7A and 7B illustrate the difference in the maneuverability of vehicle 200 between columns 4 along X axis, due to the change of the dimensions of supporting portion 208 along Y axis. In FIG. 7B, the columns 4 are presented in a dashed line in order to illustrate that when the supporting portion 208 is in its lowered position and extended state, the vehicle 200 cannot enter into the storage cell 2, because the projecting areas 214 are disposed at the location of the columns 4, and they would collide with each other.

The distance between the columns 4 along axis Y is greater than E2 and smaller than E1. This allows the vehicle 200 with its first outline 210, on one hand, to pass between the columns 4 in the retracted state of the supporting portion 208 when moving along the Y axis. On the other hand, the vehicle 200 is not able to pass between the columns 4 with its second outline 212, in the extended state of the supporting portion 208.

The columns 4 have lower portions 41 characterized by a minimal length dimension therebetween R1 in the Y axis, and the following condition is fulfilled: E2<R1<E1. The distance R1 between the lower portions 41 of the columns 2 along Y axis is related to the dimensions of the vehicle 200 in the lowered position of the supporting portion 208, so that this condition assures that in the lowered position and the retracted state of the supporting portion 208, the vehicle 200 can pass between the columns 2. According to this condition, in the lowered position and the extended state of the supporting portion 208, the vehicle 200 cannot pass between the columns 4.

The columns 4 have upper portions 42 characterized by a minimal length dimension therebetween R2 in Y axis. The distance R2 is related to a distance between bearing portions 43 of the columns 4, which best seen in FIG. 5E. Bearing portions 43 are horizontal flat surfaces, located at the upper portions 42 of the columns 4, and are configured for supporting the container thereon, while stored at the storage cell 2. Therefore, the location of the bearing portions 43 of the columns 4 is dictated by the base portions of the container, while stored within the storage cell 2.

The columns 4 are straight elongated shape, so that the following condition is fulfilled: R1=R2. According to the example of straight elongated columns, the peripheral dimensions of the storage cell 2 can be not greater than the peripheral dimensions of a container 60 stored therein, and this allows designing a storage system 1 in which containers 60 are disposed in proximity to each other, as can be seen in FIG. 1.

As shown in FIGS. 7C and 7D, the lower portions 41 are characterized by a minimal length dimension therebetween R1′, and the upper portions 42 are characterized by a minimal length dimension R2′, both in the X axis. The relationship between the parameters E1′, E2′, R1′ and R2′ is similar in a respective manner to the relationship between the parameters E1, E2, R1 and R2.

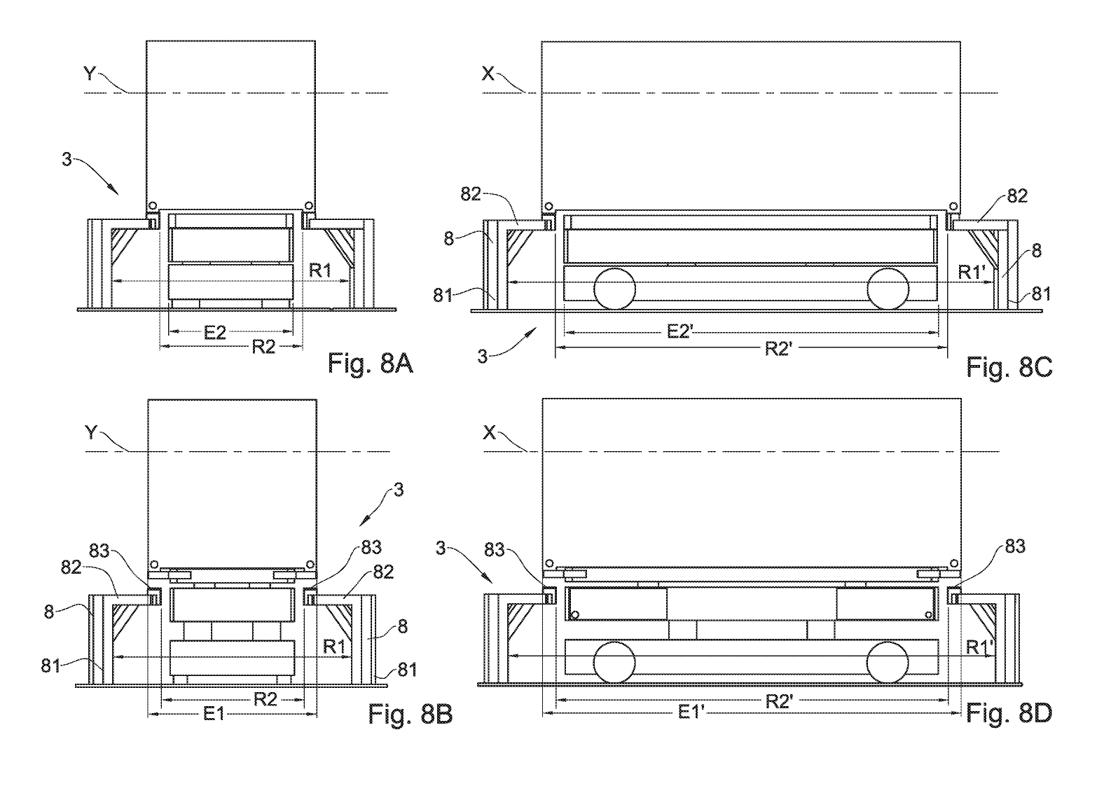

Another example of a storage cell according to the presently disclosed subject matter is a storage cell 3, illustrated in FIGS. 8A to 8D. The storage cell 3 is configured with columns 8, which are r-shaped.

The columns 8 have lower portions 81 characterized by a minimal length dimension therebetween R1 in the first axis, i.e. Y axis in the drawings.

The columns 8 further have upper portions 82 characterized by a minimal length dimension therebetween R2 in Y axis. The distance R2 is related to a distance between bearing portions 83 of the columns 8, which best seen in FIG. 8B. The bearing portions 83 are horizontal flat surfaces, located at the upper portions 82 of the columns 8, and are configured for supporting the container thereon, while stored at the storage cell 3. Therefore, the location of the bearing portions 83 of the columns 8 is configured to fit the base portions of the container, while stored within the storage cell 3.

The upper portions 82 of the columns protrude into an interior space of their storage cell 3 with respect to the lower portions 81, so that the following condition is fulfilled: R1>R2.

In addition, the following condition is fulfilled: E2<R2<E1, so that in the retracted state of the supporting portion, movement of the supporting portion 208 between its elevated position and its lowered position is allowed. According to the specific example, in the extended state of the supporting portion 208, movement of the supporting portion between its elevated position and its lowered position is prevented.

Storage cell 3 can provide storage services to both vehicles 100 and 200, with the difference that the storage cells 2 allow much compact storage structures.

The vehicle 200 also comprises an auxiliary arrangement generally designated 230. The auxiliary arrangement 230 is configured for facilitating in freeing the bottom corner fittings 61 of the container 60 during the placement of the container 60 on the columns 4. This operation is done by holding the container 60 to a short period of time, instead of the supporting portion 208, for allowing the supporting portion 208 to shift between its extended state and its retracted state, which afterwards allows placing the container within the storage cell 2 on the bottom corner fittings 61.

The auxiliary arrangement 230 is essential when the same base portions (e.g., the base corner fittings 61) are used for supporting the container 60 by the supporting portion 208 and for supporting it by the bearing portions 43 of the storage cell 2.

In the present case, during its operation, the auxiliary arrangement 230 grasps the container 60 from alternative base portions 62, best seen in FIG. 6E, which are located at the bottom corner fittings 61, so as to free the lower portion of the bottom corner fittings 61 and then placing them on the bearing portions 43 of the storage cell 2. The auxiliary arrangement 230 comprises grasping elements 232 and a secondary lifting mechanism 234, in the form of four pistons. The grasping elements 232 are configured for assuming a grasping state, seen in FIG. 6B, for grasping the container from the alternative base portions 62. By replacing the supporting portion 208 in supporting the container 60, movement of the supporting portion 208 with respect to the container 60 is allowed by the secondary lifting mechanism 234. Once the supporting portion 208 ceases to maintain contact with the container 60 it can be shifted into its retracted state.

The grasping elements 232 are also configured to assume a folded state, seen in FIGS. 6A and 6C, associated with the first outline 210 of the vehicle when seen from above.

The auxiliary arrangement further comprises a middle portion 236 disposed under the supporting portion 208 and between the primary lifting mechanism 206 and the second lifting mechanism 234. The grasping elements 232 are mounted to the middle portion 236.

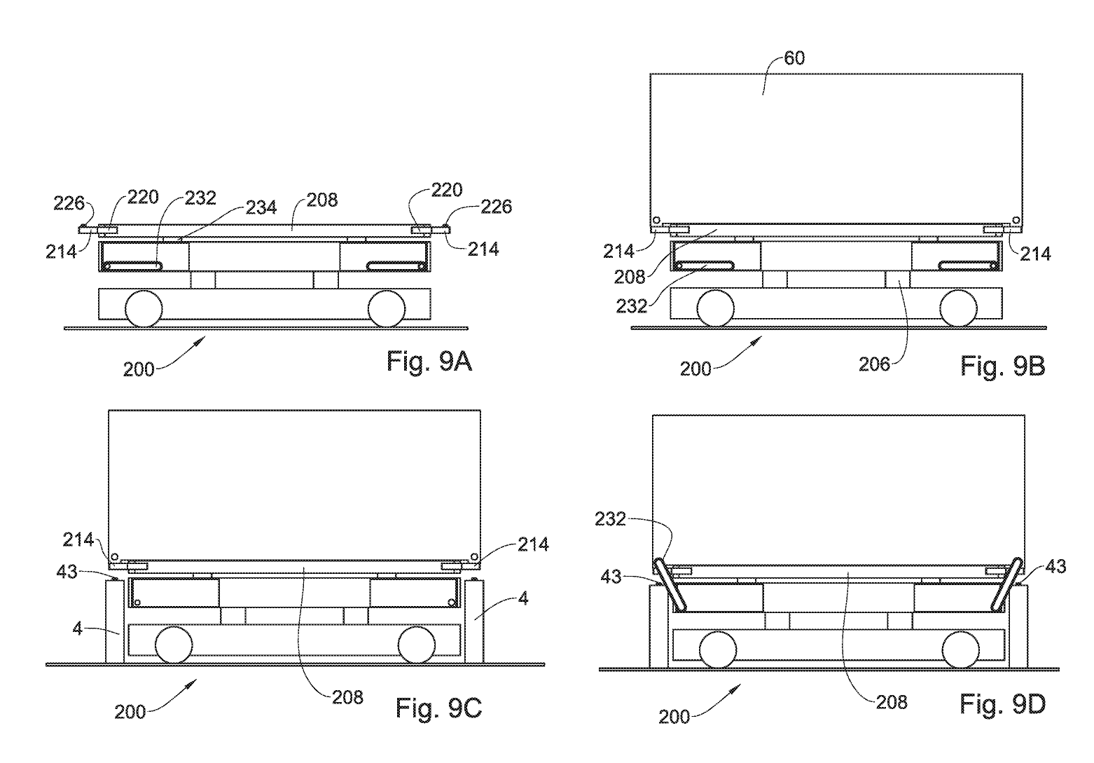

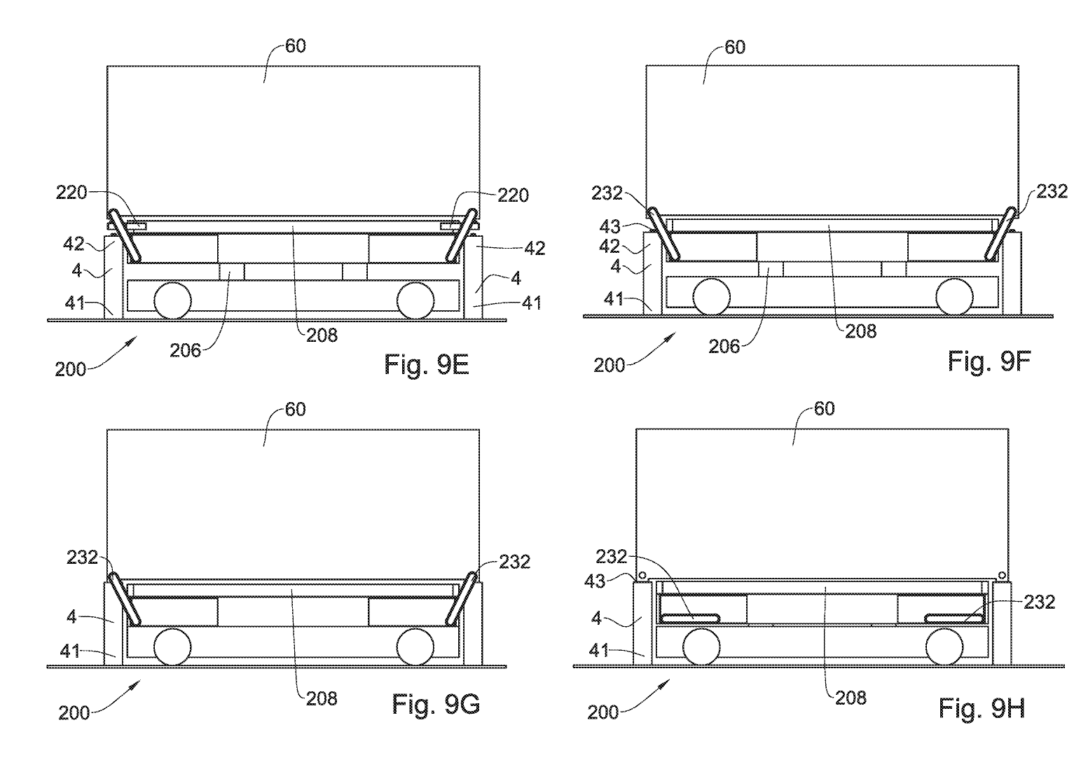

Referring now to FIGS. 9A to 9H, there are illustrated steps of inserting a container 60 into a storage cell 2 according to a method in accordance with the storing unit 80.

In FIG. 9A, the vehicle 200 is with the elevated position and the extended state of the supporting portion 208, and the folded state of the grasping elements 232. Once in that state, the vehicle 200 is ready for receiving the container 60.

FIG. 9B illustrates loading of the container 60 on the supporting portion 208, by placing the base portions, i.e., the base corner fittings 61, of the container 60 on the projecting areas 214 of the supporting portion 208. After loading the container 60 onto the supporting portion 208, the container 60 is securely held thereon by the securing lugs 260, which facilitate securing arrangement, preventing unintentional movement of the container 60 with respect to the supporting portion 208. With the container 60 secured to the supporting portion 208 by the securing lugs 260, the vehicle 200 can transport the container into the storage cell.

Even though the vehicle 200 can transport the container 60 in both the lowered and the elevated positions of the supporting portion 208, entrance into the storage cell 2 is possible only with the supporting portion 208 in its elevated position, as best seen in FIG. 9C. In this position, the retractable elements 220 are elevated higher than the bearing portions 43, and entrance of the vehicle 200 with the container 60 thereon is facilitated.

Apart from the projecting areas 214, which have outermost points spaced from each other to the distances E1 and E1′, other parts of the vehicle 200 do not project from the first outline 210 of the vehicle, i.e. their outermost points are spaced from each other to the distances E2 and E2′. As a result of that, passage of the vehicle between the lower portions of the columns, which are spaced from each other in a distance R1, is facilitated.

Moreover, having the ability to transport along both axes X and Y, and due to its dimensions, the vehicle 200 can enter the cell 2 from all four directions, i.e. along axis X and along axis Y.

As shown in FIG. 9C, the vehicle 200 supports the container 60 from the bottom corner fittings 61, and therefore, locating the same bottom corner fittings 61 is not allowed when the supporting portion 208 is in its extended state. For enabling transferring the container from the projecting areas 214 of the supporting portion 208 to the bearing portions 43 of the columns 4, the supporting portion 208 has to be shifted into its retracted state, for exposing the bottom corner fittings 61 and thereby to allow placing them on the bearing portions 43. For that purpose, as seen in FIG. 9D, the grasping elements 232 are shifted into their grasping state, and replace the retractable elements 220 of the supporting portion 208 in supporting the container 60. The grasping elements 232 support the container 60 from the alternative base portions 62.

Consequently, movement of the supporting portion 208 by the secondary lifting mechanism 234 with respect to the container 60, which is now being held by the middle portion 236 via the grasping elements 232, is allowed. FIG. 9E shows how the supporting portion 208 is lowered with respect to the container 60. Now, shifting of the supporting portion 208 to its retracted state is allowed.

In the retracted state of the supporting portion 208, the bottom corner fittings 61 are exposed with respect to the bearing portions 43, and are ready to be placed thereon, as seen in FIG. 9F.

The next step, as illustrated in FIG. 9G, is moving the supporting portion 208 to its lowered position by the primary lifting mechanism 206, thereby placing the container 60 on the bearing portions 43 of the columns 4.

Once supported by the bearing portions 43 of the columns 4, shifting the grasping elements 232 to their folded state is enabled, and thereby the vehicle 200 is in its most compact state, and is ready for escaping from the storage cell 2.

FIG. 9H shows the vehicle 200 in its compact state, ready for exiting the storage cell 2 in the desired direction, i.e. along X axis or along Y axis.

The method can further comprise steps of taking a container 60 out from a storage cell 2. These steps are reverse steps to the steps of insertion of a container 60 into the storage cell 2, and are performed in a reversed manner.

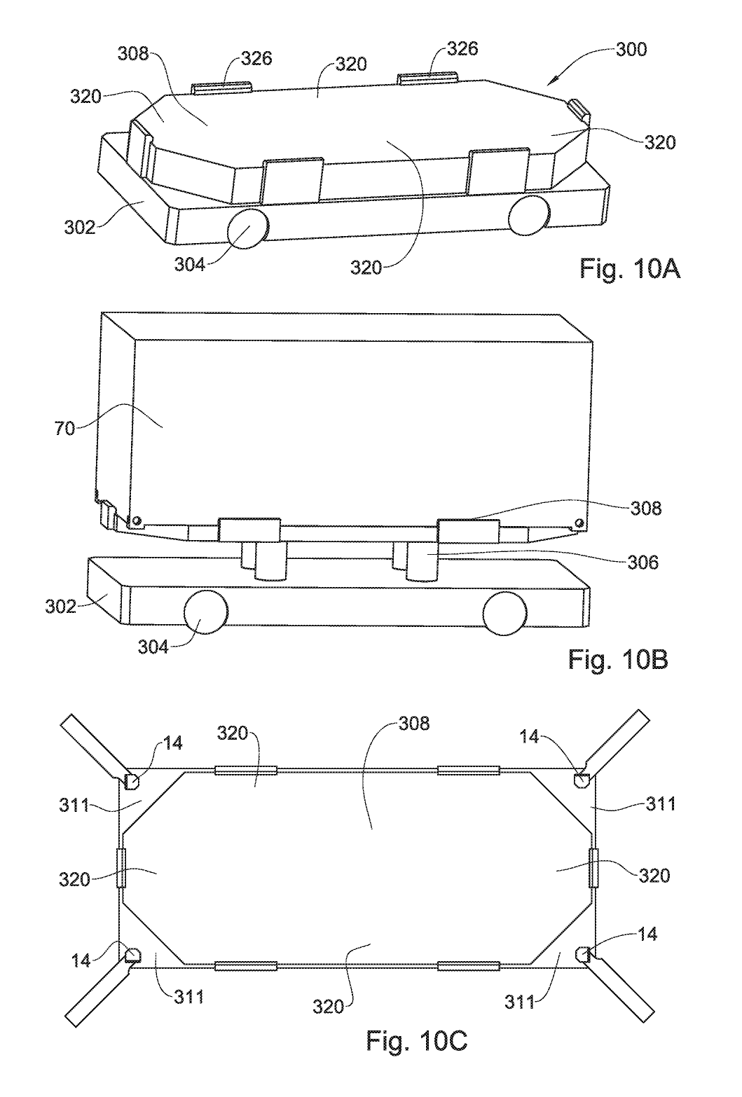

Another example of a vehicle according to the presently disclosed subject matter is vehicle 300, illustrated in FIGS. 10A to 15.

The vehicle 300 comprises a body 302, and a propulsion arrangement 304 mounted thereto. The propulsion arrangement 304 is configured with omnidirectional wheels, allowing the vehicle 300 to transport along a first axis of the vehicle, namely Y axis, and a second axis thereof, namely X axis. Axes Y and X are perpendicular to each other.

The vehicle 300 is configured with a supporting portion 308, which is elevatable with respect to the body 302 via a primary lifting mechanism 306, seen in FIG. 10B, in the form of four pistons. The lifting mechanism 306 is configured to move the supporting portion 308 between an elevated position, seen in FIG. 10B, and a lowered position, seen in FIG. 10A.

The supporting portion 308 comprises supporting areas 320 configured for supporting the container 70 at base portions thereof. As can be understood from the drawings, the supporting areas 320 of the supporting portion 308 are configured for supporting the container 70 from its load transfer areas 72 (which are similar to the load transfer areas 52 of the container 50) and not from the bottom corner fittings 71 (which are similar to the bottom corner fittings 51 of the container 50), and therefore, vehicle 300 is configured for carrying containers other than 1D and 1DX.

The supporting portion 308 is structured with exposed portions 311 at its corners, seen in FIG. 10C, so that when a container is received thereon, additional base portions of the container are exposed, when seen from below. The additional base portions of the container are its bottom corner fittings 71, which are exposed when seen from below (FIG. 12), and capable of being placed on respective bearing portions of a storage cell, as will be described below, when the supporting portion 308 is moved from its elevated position to its lowered position.

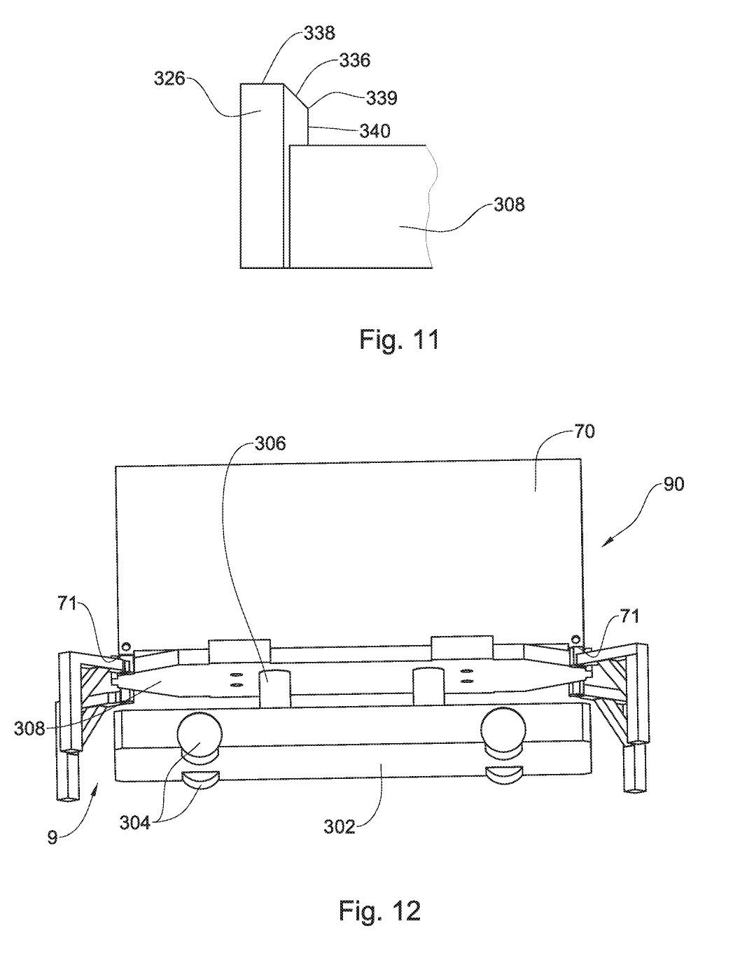

The supporting portion 308 is configured with a guiding-securing arrangement, in the form of guiding-securing projections 326, seen in enlarged view in FIG. 11. The guiding-securing projections 326 are mechanically associated the said supporting portion 308 and configured for guiding the container 70 during loading thereof on the vehicle 300, for properly locating the container 70 with respect to the supporting portion 308, and securing the container 70 to the supporting portion 308 so as to prevent the container 70 from moving with respect thereto at least during transportation of the container 70 by the vehicle 300.

The guiding of the container is gained by the shape of the guiding-securing projections 326, seen best in FIG. 11. Each guiding-securing projection 326 has an inclined surface 336, which is configured to guide the container 70 while being loaded to the vehicle 300 and for properly locating the container 70 with respect to the supporting portion 308, i.e. guide the load transfer areas 72 of the container to place on the supporting areas 320 of the supporting portion 308. During the loading of the container 70 on the supporting portion 308, the operator in charge of the loading can locate the container between distal ends 338 of the biased surfaces 336, and then slide the container downwardly along the biased surfaces 336 of the guiding-securing projections 326 to the proper location on the supporting portion 308. The locations of the proximate ends 339 of the biased surfaces 336 facilitate proper location of the container 70 on the supporting portion 308.

In addition, the guiding-securing projections 326 constitute securing elements, preventing unintentional movement of the container 70 with respect to the supporting portion 308. The securing effect is gained by the protrusion of the guiding-securing projections 326, which act as stopping elements, preventing the container from moving. As best seen in FIG. 11, each guiding-securing projection 326 is configured with an edge 340, limiting the container 70 from moving horizontally when placed on the supporting portion 308. This arrangement facilitates transportation of the vehicle 300 with the container 70 thereon, in both the elevated and lowered positions of the supporting portion 308.

The vehicle 300 is adapted to operate in conjunction with storage cell 9, for storing a container 70 therein.

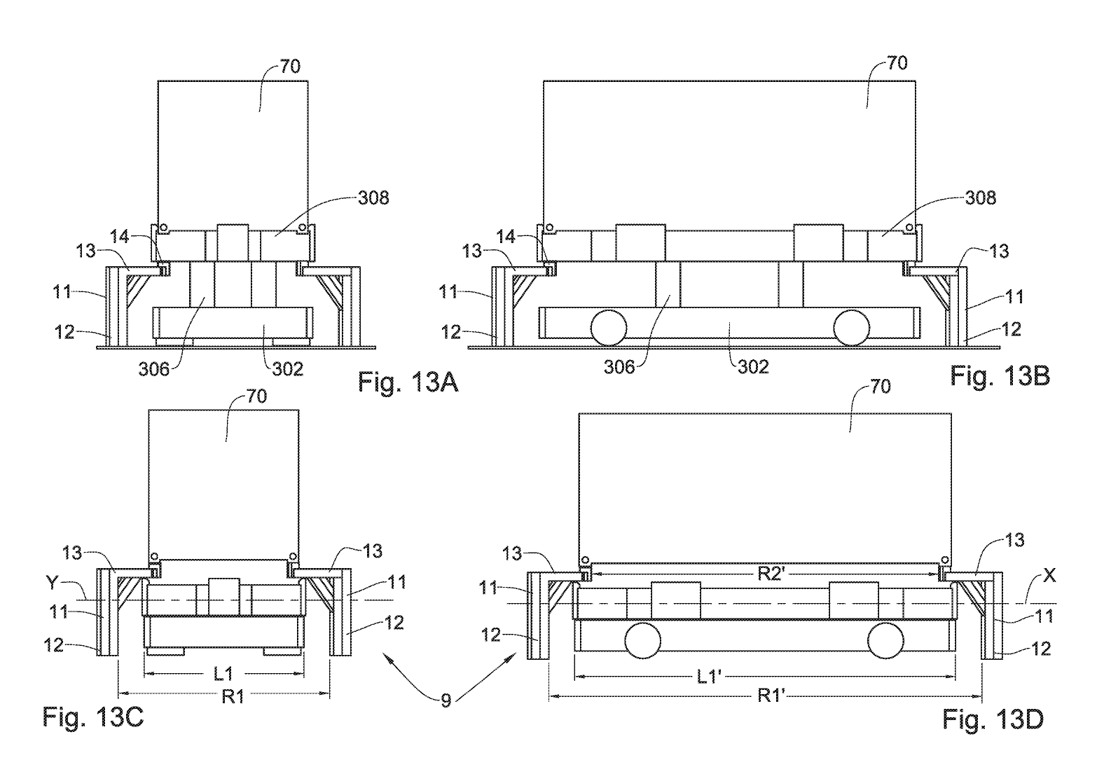

The vehicle 300 has a first maximal length dimension (L1) in the first axis, i.e. Y axis. The columns 11 comprise lower portions 12 characterized by a minimal length dimension in the first axis associated with the distance therebetween (R1) and the following condition is fulfilled: R1>L1, so that transportation of the vehicle along the second axis, i.e. X axis, into and from the storage cell, at least in the lowered position of the supporting portion without said container thereon is allowed (FIGS. 13A and 13C).

The columns 11 further comprise upper portions 13 characterized by a minimal length dimension in the second axis associated with the distance therebetween (R2) and the upper portions protrudes into an interior space of the storage cell with respect to the lower portions 12, so that the following condition is fulfilled: R1>R2. The upper portions comprise bearing portions 14, configured for supporting the container 70 from its bottom corner fittings 71.

The supporting portion has a first maximal length dimension (L1′) in the second axis, i.e. X axis, and the lower portions 12 of the columns 11 are characterized by a minimal length dimension in the second axis associated with the distance therebetween (R1′). the following condition is fulfilled: R1′>L1′, so that transportation of the vehicle 300 along Y axis into and from the storage cell, in the lowered position of the supporting portion 308 without the container 70 thereon is allowed (FIGS. 13B and 13D).

As can be seen in FIGS. 13A and 13B, the vehicle can transport the container 70 into and from the storage cell 9 in the elevated position of the storage cell, in such a manner that the supporting portion 308 is elevated higher than the bearing portions 14, of the columns 11, along both axes, X and Y.

When the vehicle 300 is inside the storage cell 9, and with the elevated position of the supporting portion 308, the exposed portions 311 of the supporting portion 308 are located above the columns 11, and more particularly above the bearing portions 14, thereby allowing movement of the supporting portion 308 to its lowered position, by the primary lifting mechanism 306.

Once the vehicle 300 is located inside the storage cell 9, the supporting portion 308 can be moved between the elevated position, seen in FIGS. 13A and 13B, to its lowered position, seen in FIGS. 13C and 13D, and thereby transferring the load of the container 70 from the supporting portion 308 to the bearing portions 14 of the storage cell 9.

1. A vehicle configured for conveying at least one container, said vehicle having a supporting portion configured for holding said container, and a primary lifting mechanism for controlling elevation of the supporting portion, said lifting mechanism being configured to move the supporting portion between an elevated position and a lowered position; said supporting portion being configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion; the supporting portion in its extended state having projecting areas, projecting in the second outline relative to the first outline and configured for supporting said container.

2. The vehicle according to claim 1, wherein the projecting areas have outermost points spaced from each other to a distance D1 defining a maximal dimension of the second outline along a first axis of the vehicle, and said first outline has outermost points spaced from each other to a distance D2 defining a maximal dimension along said first axis, which is smaller than D1, allowing the vehicle with its first outline to pass, along a second axis perpendicular to said first axis, between two columns spaced from each other along said first axis to a distance greater than D2 and smaller than D1, without said container thereon.

3. The vehicle according to claim 2, wherein in said extended state of the supporting portion, the projecting areas extend at least in said first axis to support the container at respective base portions thereof.

4. The vehicle according to any one of the preceding claims, wherein each of said projecting areas is associated with a corresponding retractable element that is retracted in the retracted state of the supporting portion and is extended in the extended state of the supporting portion.

5. The vehicle according to claim 4, wherein said supporting portion comprises a plurality of recesses, each configured at least partially to accommodate at least one of said retractable elements in the retracted state of the supporting portion.

6. The vehicle according to any one of the preceding claims, wherein the projecting areas comprise a securing arrangement configured for securing the container to the supporting portion, thereby preventing the container from moving with respect to the supporting portion at least during transportation of the container by the vehicle.

7. The vehicle according to claim 6, wherein at least part of the projecting areas comprise a guiding arrangement configured for guiding the container during loading thereof on the vehicle for properly locating the container with respect to the supporting portion.

8. The vehicle according to claim 7, wherein said securing arrangement and said guiding arrangement are integrated in a common securing-guiding arrangement.

9. The vehicle according to claim 7 or 8, wherein at least one of said securing arrangement and said guiding arrangement is configured to assume a folded unoperative position and an unfolded operative position.

10. The vehicle according to any one of the preceding claims, wherein said supporting portion has a rectangular shape defined by four corners, and said projecting areas are disposed at said corners.

11. The vehicle according to any one of claims 1 to 9, wherein said supporting portion has a rectangular shape defined by four corners with sides therebetween, and said projecting areas are disposed at least part of said sides.

12. The vehicle according to any one of the preceding claims, wherein the vehicle comprises an auxiliary arrangement configured for holding the container instead of said supporting portion for allowing the supporting portion to shift between its extended state and its retracted state.

13. The vehicle according to claim 12, when dependent on claim 3, wherein the auxiliary arrangement comprises one or more grasping elements and a secondary lifting mechanism; said grasping elements being configured for assuming a grasping state for grasping the container from alternative base portions other than said base portions, thereby allowing movement of the supporting portion or the container with respect to each other by the secondary lifting mechanism so as to allow shifting of the supporting portion between its extended state and its retracted state, and a folded state associated with the first outline of the vehicle when seen from above.

14. The vehicle according to claim 13, wherein the auxiliary arrangement further comprises a middle portion disposed under said supporting portion and between the primary lifting mechanism and the second lifting mechanism.

15. The vehicle according to claim 14, wherein said grasping elements are mounted to said middle portion.

16. The vehicle according to any one of the preceding claims, wherein said supporting portion is constituted by a plurality of supporting portions, each associated with a different portion of the vehicle.

17. The vehicle according to any one of the preceding claims, wherein the supporting portion and the primary lifting mechanism are add-ons mountable on a body of a vehicle.

18. The vehicle according to any one of the preceding claims, when dependent on claim 2, wherein the projecting areas have outermost points spaced from each other to a distance D1′ defining a maximal dimension of the second outline along the second axis of the vehicle, and said first outline has outermost points spaced from each other to a distance D2′ defining a maximal dimension along the second axis, which is smaller than D1′, allowing the vehicle with its first outline to pass, along the first axis perpendicular to said second axis, between columns of a cell spaced from each other along said second axis to a distance greater than D2′ and smaller than D1′, without said container thereon and, when having the second outline, to locate said container on the bearing portions of the columns of said cell

19. A system for storage of at least one container, comprising:
a. a storage structure having a plurality of storage cells, each comprising a plurality of columns with bearing portions configured to support said container within said storage cell;
b. at least one vehicle, configured for conveying said container to and from the storage cell, said vehicle having a supporting portion configured for holding said container, and a primary lifting mechanism for controlling elevation of the supporting portion, said lifting mechanism being configured to move the supporting portion between its elevated position and its lowered position; said supporting portion being configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion; the supporting portion in its extended state having projecting areas, projecting in the second outline relative to the first outline and configured for supporting said container.

20. The system according to claim 19, wherein the projecting areas have outermost points spaced from each other to a distance D1 defining a maximal dimension of the second outline along a first axis of the vehicle, and said first outline has outermost points spaced from each other to a distance D2 defining a maximal dimension along said first axis, which is smaller than D1, allowing the vehicle with its first outline to pass, along a second axis perpendicular to said first axis, between columns of a cell spaced from each other along said first axis to a distance greater than D2 and smaller than D1, without said container thereon and, when having the second outline, to locate said container on the bearing portions of the columns of said cell.

21. The system according to claim 20, wherein said columns comprise lower portions characterized by a minimal length dimension in the first axis associated with the distance therebetween (R1) and the following condition is fulfilled: D2<R1<D1, so that at least in the retracted state of the supporting portion, transportation of the vehicle along said second axis into said storage cell, in the lowered position of the supporting portion without said container thereon is allowed, and in said extended state of the supporting portion, said transportation is prevented.

22. The system according to claim 21, wherein said columns comprise upper portions characterized by a minimal length dimension in the first axis associated with the distance therebetween (R2) and the following condition is fulfilled: D2<R2<D1, so that at least in the retracted state of the supporting portion, movement of the supporting portion between its elevated position and its lowered position is allowed.

23. The system according to claim 22, wherein said conditions: D2<R2<D1 are fulfilled, so that at least in the extended state of the supporting portion, movement of the supporting portion between its elevated position and its lowered position is prevented.

24. The system according to any one of claim 22 or 23, wherein said columns are have straight elongated shape, and the following condition is fulfilled: R1=R2, and the bearing portions of the columns are horizontal flat surfaces.

25. The system according to any one of claim 22 or 23, wherein said upper portions protrude into an interior space of their storage cell with respect to the lower portions, so that the following condition is fulfilled: R1>R2.

26. The system according to any one of claims 20 to 25, wherein the projecting areas have outermost points spaced from each other to a distance D1′ defining a maximal dimension of the second outline along the second axis of the vehicle, and said first outline has outermost points spaced from each other to a distance D2′ defining a maximal dimension along the second axis, which is smaller than D1′, allowing the vehicle with its first outline to pass, along the first axis perpendicular to said second axis, between columns of a cell spaced from each other along said second axis to a distance greater than D2′ and smaller than D1′, without said container thereon and, when having the second outline, to locate said container on the bearing portions of the columns of said cell.

27. The system according to claim 26, wherein said columns comprise lower portions characterized by a minimal length dimension in the second axis associated with the distance therebetween (R1′) and the following condition is fulfilled: D2′<R1′<D1′ and R1′>D2′, so that at least in the retracted state of the supporting portion, transportation of the vehicle along said second axis into said storage cell, in the lowered position of the supporting portion without said container thereon is allowed, and in said extended state of the supporting portion, said transportation is prevented.

28. The system according to claim 27, wherein said columns comprise upper portions characterized by a minimal length dimension in the second axis associated with the distance therebetween (R2′) and the following condition is fulfilled: D2′<R2′<D1′, so that at least in the retracted state of the supporting portion, movement of the supporting portion between its elevated position and its lowered position is allowed.

29. The system according to claim 28, wherein said conditions: D2′<R2′<D1′ are fulfilled, so that at least in the extended state of the supporting portion, movement of the supporting portion between its elevated position and its lowered position is prevented.

30. A system for storage of at least one container, comprising:
a. a storage structure having a plurality of storage cells, each comprising a plurality of columns with configured to support said container within said storage cell;
b. at least one vehicle configured for conveying said container to and from the storage cell, said vehicle having a supporting portion configured for holding said container, and a primary lifting mechanism for controlling elevation of the supporting portion, said lifting mechanism being configured to move the supporting portion between its elevated position and its lowered position;
c. a guiding-securing arrangement mechanically associated with said supporting portion and configured for: guiding the container during loading thereof on the vehicle for properly locating the container with respect to the supporting portion, and securing the container to the supporting portion so as to prevent the container from moving with respect thereto at least during transportation of the container by the vehicle.

31. The system according to claim 30, wherein said supporting portion comprises supporting areas configured for supporting said container at base portions thereof, and wherein said supporting portion is structured so that when the container is received thereon, additional base portions of the container are exposed, when seen from below, for being placed on said bearing portions when the supporting portion is moved from its elevated position to its lowered position.

32. The system according to claim 30 or 31, wherein the vehicle has a first axis and a second axis perpendicular thereto and the supporting portion has a first maximal length dimension (L1) in said first axis, and wherein said columns comprise lower portions characterized by a minimal length dimension in the first axis associated with the distance therebetween (R1) and the following condition is fulfilled: R1>L1, so that transportation of the vehicle along said second axis into said storage cell, at least in the lowered position of the supporting portion without said container thereon is allowed.

33. The system according to claim 32, wherein said columns comprise upper portions characterized by a minimal length dimension in the second axis associated with the distance therebetween (R2) and said upper portions protrude into an interior space of their storage cell with respect to the lower portions, so that the following condition is fulfilled: R1>R2.

34. The system according to any one of claim 31 or 32, wherein the supporting portion has a first maximal length dimension (L1′) in said second axis, and wherein said columns comprise lower portions characterized by a minimal length dimension in the second axis associated with the distance therebetween (R1′) and the following condition is fulfilled: R1′>L1′, so that transportation of the vehicle along said first axis into said storage cell, in the lowered position of the supporting portion without said container thereon is allowed.

35. A method for using a system for storage of at least one container, comprising steps of:
a. providing said system for storage of at least one container, comprising:
a storage structure having a plurality of storage cells, each comprising a plurality of columns with bearing portions; and
at least one vehicle, having a supporting portion and a primary lifting mechanism configured to move the supporting portion between an elevated position and a lowered position; said supporting portion being configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion; the supporting portion in its extended state having projecting areas, projecting in the second outline relative to the first outline and configured for supporting said container;
b. shifting said supporting portion to its extended state;
c. loading the container on said supporting portion by placing base portions of the container on said projecting areas;
d. transporting the vehicle with the container thereon into the storage cell while the supporting portion is disposed in its elevated position;
e. moving said supporting portion to its lowered position by said primary lifting mechanism, thereby placing the container on said bearing portions;
f. shifting the supporting portion to its retracted state; and
g. transporting the vehicle without the container thereon from the storage cell.

36. The method according to claim 35, further comprising steps of:
h. transporting the vehicle to the storage cell while a container is stored in the storage cell and the supporting portion is in its lowered position and its retracted state;
i. shifting the supporting portion to its extended state;
j. moving said supporting portion to its elevated position by said primary lifting mechanism, thereby placing the container on said projecting areas;
k. transposing the vehicle with the container thereon from the storage cell.

37. A method for using a system for storage of at least one container, comprising steps of:
a. providing said system for storage of at least one container, comprising:
a storage structure having a plurality of storage cells, each comprising a plurality of columns with bearing portions; and
at least one vehicle, having a supporting portion; an auxiliary arrangement; and a primary lifting mechanism configured to move the supporting portion between an elevated position and a lowered position; said supporting portion being configured for assuming a retracted state associated with a first outline of the vehicle when seen from above, and combinable at least with the lowered position of the supporting portion, and an extended state associated with a second outline of the vehicle when seen from above and combinable at least with the elevated position of the supporting portion; the supporting portion in its extended state having projecting areas, projecting in the second outline relative to the first outline and configured for supporting said container; said auxiliary arrangement comprising one or more grasping elements and a secondary lifting mechanism; said grasping elements being configured for assuming a grasping state and a folded state associated with the first outline of the vehicle when seen from above;
b. shifting said supporting portion to its extended state;
c. loading the container on said supporting portion by placing base portions of the container on said projecting areas;
d. transporting the vehicle with the container thereon into the storage cell while the supporting portion is disposed in its elevated position;
e. shifting said grasping elements to their grasping state, thereby grasping the container from alternative base portions other than said base portions;
f. moving of the supporting portion or the container with respect to each other by the secondary lifting mechanism;
g. shifting the supporting portion to its retracted state;
h. moving said supporting portion to its lowered position by said primary lifting mechanism, thereby placing the container on said bearing portions;
i. shifting said grasping elements to their folded state; and
j. transporting the vehicle without the container thereon from the storage cell.

38. The method according to claim 37, further comprising steps of:
k. transporting the vehicle to the storage cell while a container is stored in the storage cell and the supporting portion is in its lowered position and its retracted state;
l. shifting said grasping elements to their grasping state, thereby grasping the container from alternative base portions other than said base portions;
m. moving said supporting portion to its elevated position by said primary lifting mechanism;
n. shifting the supporting portion to its extended state;
o. moving of the supporting portion or the container with respect to each other by the secondary lifting mechanism, thereby placing the container on said projecting areas;
P. shifting said grasping elements to their folded state;
q. transposing the vehicle with the container thereon from the storage cell.
